(19) United States
(12) Patent Application Publication
(10) Pub. No.: US 2017/0290718 A1
(43) Pub. Date: Oct. 12, 2017

(54) Top Sheet For Absorbent Articles

(71) Applicant: OJI HOLDINGS CORPORATION, Tokyo (JP)

(72) Inventors: Yoshihiro TAKIYAMA, Tokyo (JP); Izumi TASHIRO, Tokyo (JP); Katsuhiko SUGIYAMA, Tokyo (JP)

(73) Assignee: OJI HOLDINGS CORPORATION, Tokyo (JP)

(21) Appl. No.: 15/507,733

(22) PCT Filed: Aug. 26, 2015
(86) PCT No.: PCT/JP2015/073968

§ 371 Date: Feb. 28, 2017

(30) Foreign Application Priority Data
Sep. 2, 2014 (JP) 2014-177689
Jan. 28, 2015 (JP) 2015-013994
Jan. 28, 2015 (JP) 2015-013995
Jan. 28, 2015 (JP) 2015-013996
Jan. 28, 2015 (JP) 2015-014639

(51) Int. Cl. A61F 13/511; A61F 13/49

(52) U.S. Cl. CPC A61F 13/511; A61F 13/49; A61F 13/15

(57) ABSTRACT

The present invention addresses the problem of improving quick-drying properties of a top sheet at a part that often contacts with the skin of a wearer. A top sheet (10) for absorbent articles comprises an upper layer sheet (11), a lower layer sheet (12), multiple bonded parts (40) at which the sheets (11, 12) are bonded to each other, and multiple unbonded parts (50) which are surrounded by the multiple bonded parts (40). Each of the unbonded parts (50) has a larger area (51) that has a relatively large area and a smaller area (52) that has a relatively small area and is extended in one direction from the larger area (51). The upper layer sheet (11) is protruded convexly in the larger areas (51) and the smaller areas (52).

### TECHNICAL FIELD

The present invention relates to a top sheet provided on a surface of an absorbent article directly coming into contact with a skin of a wearer. More specifically, the invention relates to a top sheet obtained by joining a plurality of stacked sheet members using embossing.

### BACKGROUND ART

A tape-type disposable diaper, a pants-type disposable diaper, a urine pad, a panty liner, a light incontinence pad, a sanitary napkin, etc. has been known as an absorbent article installed on a crotch of a wearer. In such an absorbent article, a surface facing a skin of the wearer (skin facing surface) includes a top sheet. The top sheet is a sheet member that continuously comes into contact with the skin of the wearer. Thus, in general, a material causing low skin irritation is used. For example, a material causing low irritation is considered to be preferable for the top sheet. Examples of the irritation include mechanical irritation generated due to contact or friction with the skin, irritation due to environmental deterioration such as mustiness at a position at which the absorbent article is worn, chemical irritation generated when a hydrophilizing agent used for the top sheet or excreta touches the skin, etc.

In addition, a conventionally known scheme, an uneven embossing pattern is formed by performing embossing on the top sheet for the absorbent article to reduce a contact area with the skin, thereby reducing mechanical irritation due to friction, etc. In addition, since a gap is generated between the skin and the top sheet when embossing is performed on the top sheet, it is possible to expect an effect that mustiness is partially relieved. Further, when embossing is performed on the top sheet, excreta such as urine flows along unevenness and easily diffuses, and absorbency of the top sheet is considered to be improved by diffusion effect thereof. In this way, various embossing patterns formed on the top sheet have been devised in order to further improve a low irritation property, a mustiness relief effect, and a liquid diffusion effect of the top sheet (Patent Literatures 1 to 3).

### CITATION LIST

### Patent Literature

Patent Literature 1: JP 2009-000512 A

Patent Literature 2: JP 2004-000466 A

Patent Literature 3: JP 2009-160032 A

### SUMMARY OF INVENTION

### Technical Problem

Patent Literatures 1 to 3 disclose a conventional embossing pattern in which a region surrounded by three or more joining portions is set to a protrusion. In the conventional embossing pattern, the joining portions are formed at positions of latticed apexes of a triangle, a rectangle, or another polygon. For this reason, in the conventional embossing pattern, for example, a protrusion surrounded by a plurality of joining portions has a simple shape such as a regular triangle, a square, a regular hexagon, etc. In this way, when the protrusion of the embossing pattern has a simple shape such as a square, and when a liquid excreted by the wearer touches the top sheet, the top sheet evenly diffuses the liquid in every direction. When the liquid is evenly diffused in every direction, there is a merit that liquid absorption efficiency is enhanced.

However, when the liquid touching the top sheet is diffused all around the protrusion, a region of the top sheet touched by the liquid is evenly damp in a wide range. As a result, there has been a problem that the region damped by absorbing the liquid rarely dries as a whole. In other words, when the liquid evenly diffuses around the protrusion, the region damped by this liquid almost simultaneously dries. However, there is a desire to rapidly dry at least a region easily touching the skin of the wearer by priority when the top sheet absorbs the liquid.

In this regard, an issue to be solved by the invention is to provide a top sheet in which a quick drying property of a region that easily touches the skin of the wearer may be enhanced.

### Solution to Problem

As a result of a keen examination of solution to the above-mentioned problem, the inventors of the invention have determined to form a plurality of joining portions around a non-joining portion that convexly swells such that the non-joining portion includes a large region having a large area and a small region having a small area which extends in one direction from the large region. In this way, when a liquid touches a top sheet, the liquid may be rapidly moved from the large region to the small region, and the large region easily touched by a skin of a wearer may be rapidly dried by priority. Since the liquid flows from the large region into the small region, there is a possibility that a drying time of the small region may increase. However, it is possible to enhance at least a quick drying property of the large region. When a drying time of the large region having the large area shortens, a feeling of wearing an absorbent component including the top sheet is improved. In addition, the inventors have conceived that the problem of the conventional invention may be solved based on the above-described information, and completed the invention. Specifically, the invention has configurations below.

A first aspect of the invention relates to a top sheet 10 of an absorbent article.

The top sheet 10 of the invention includes an upper layer sheet 11, a lower layer sheet 12, a plurality of joining portions 40, and a plurality of non-joining portions 50.

The upper layer sheet 11 and the lower layer sheet 12 are stacked on each other. The upper layer sheet 11 is positioned on a side that directly touches the skin of the wearer, and the lower layer sheet 12 is disposed on a rear side of the upper layer sheet 11, and does not directly touch the skin of the wearer.

The plurality of joining portions 40 joins the upper layer sheet 11 and the lower layer sheet 12 to each other.

Each of the plurality of non-joining portions 50 is surrounded by a plurality of joining portions 40.

Herein, each of the non-joining portions 50 includes a large region 51 having a relatively large area, and a small region 52 having a relatively small area and extending in one direction from the large region 51. The upper layer sheet 11 convexly swells in the large region 51 and the small region 52.

The large region 51 and the small region 52 may hold spaces therein, and insides thereof may be filled with fiber.

As in the above-described configuration, in the invention, the non-joining portion 50 surrounded by the plurality of joining portions 40 has a shape that includes only the large region 51 having the large area and the small region 52 having the small area. In this way, the liquid touching the top sheet 10 may be rapidly moved from the large region 51 to the small region 52. For example, in order to shorten a drying time of the liquid, the large region 51 may be swelled higher than the small region 52, or a fiber density (fiber filling rate) of the small region 52 may be set to be higher than that of the large region 51. In this way, it is possible to enhance a quick drying property of the large region 51 easily touched by the skin of the wearer. In particular, when the large region 51 and the small region 52 of the non-joining portion 50 have spaces therein, a repulsive force is not greatly different between the large region 51 and the small region 52, and slowly changes. Thus, a tactile property of the top sheet 10 may be softened.

In the top sheet 10 of the invention, each of the plurality of joining portions 40 preferably has a shape in which a first line portion 41, a second line portion 42, and a third line portion 43 connected to one another branch off from a junction 44 thereof in different directions. In addition, the first line portion 41 is preferably curved or bent to swell in a certain direction, and the second line portion 42 is preferably curved or bent to swell in an opposite direction to the first line portion 41. In other words, a soft S-shape (curved line shape) or Z-shape (bent line shape) is formed by the first line portion 41 and the second line portion 42.

As in the above-described configuration, when each of the joining portions 40 has a shape in which three line portions branch off in three directions, and joining portions 40 having such a shape are regularly disposed, the non-joining portion 50 may be formed. In addition, when the first line portion 41 and the second line portion 42 among the three line portions are curved or bent to swell in opposite directions, the large region 51 and the small region 52 may be formed in the above-described non-joining portion 50. In this way, in the invention, the joining portion 40 preferably has a λ-shape. In addition, in the invention, since all the plurality of joining portions 40 may have the λ-shape, and be disposed in the same direction, an appearance of the top sheet 10 is excellent.

In the top sheet 10 of the invention, three λ-shaped joining portions 40 are preferably positioned around one certain non-joining portion 50. In this instance, the three joining portions 40 are a first joining portion 40a, a second joining portion 40b, and a third joining portion 40c.

In this case, a portion around the one certain non-joining portion 50 is preferably demarcated by elements (i) to (vi) below.

(i) First line portion 41 and second line portion 42 of first joining portion 40a

(ii) Second line portion 42 and third line portion 43 of second joining portion 40b

(iii) Third line portion 43 and first line portion 41 of third joining portion 40c

(iv) First gap 61 between first line portion 41 of first joining portion 40a and third line portion 43 of second joining portion 40b

(v) Second gap 62 between second line portion 42 of second joining portion 40b and first line portion 41 of third joining portion 40c

(vi) Third gap 63 between third line portion 43 of third joining portion 40c and second line portion 42 of first joining portion 40a

When the portion around the non-joining portion 50 is demarcated as described above, the non-joining portion 50 including the large region 51 and the small region 52 may be appropriately formed.

In the top sheet 10 of the invention, a height at which the large region 51 swells is preferably higher than a height at which the small region 52 swells.

As in the above-described configuration, when the convex large region 51 is higher in height than the convex small region 52, the liquid touching the top sheet 10 may be rapidly moved from the large region 51 to the small region 52. In addition, when the large region 51 is higher than the small region 52, the skin of the wearer easily touches the large region 51 having the large area and the high height. On the contrary, the skin of the wearer rarely touches the small region 52 having the small area and the low height. Therefore, the skin of the wearer easily touches the large region 51 having a quick drying property, and is prevented from touching the small region 52 which is difficult to dry. Further, when the small region 52 is lower than the large region 51, the large region 51 is supported by the small region 52, and a cushioning property of the large region 51 is enhanced. For this reason, even when a body pressure of the wearer is applied to the top sheet 10, the large region 51 is rarely crushed. In addition, even when the large region 51 is crushed once, an original shape is easily restored.

In the top sheet 10 of the invention, the large region 51 may have a space therein, and an inside of the small region 52 may be filled with fiber.

As in the above-described configuration, when the large region 51 is formed in a dome shape having an internal space, and an inside of the small region 52 is filled with fiber included in the upper layer sheet 11, the liquid touching the top sheet 10 easily moves from the large region 51 to the small region 52. In addition, when the large region 51 is supported by the small region 52, the cushioning property of the large region 51 is enhanced.

In the top sheet 10 of the invention, the large region 51 and the small region 52 may have spaces therein.

As in the above-described configuration, in the invention, the non-joining portion 50 surrounded by the plurality of joining portions 40 has a shape including only the large region 51 having the large area and the small region 52 having the small area. In addition, the large region 51 and the small region 52 are formed in a dome shape swelled to hold a space therein. In this way, the liquid touching the top sheet 10 may be rapidly moved from the large region 51 to the small region 52, and the quick drying property of the large region 51 easily touched by the skin of the wearer may be enhanced. Further, when the large region 51 and the small region 52 of the non-joining portion 50 have spaces therein, a repulsive force is not greatly different between the large region 51 and the small region 52, and slowly changes. Thus, a tactile property of the top sheet 10 may be softened.

In the top sheet 10 of the invention, crush points 53 that press the upper layer sheet 11 toward a side of the lower layer sheet 12 are preferably formed in some of the plurality of non-joining portions 50. In this case, the non-joining portions 50 in which the crush points 53 are formed are preferably continuously disposed in a certain direction.

As in the above-described configuration, a plurality of joining portions 40 is regularly formed by performing embossing on the top sheet 10, and a non-joining portion 50 surrounded by the joining portions 40 is convexly swelled to hold a space therein. However, in a pattern in which a plurality of non-joining portions 50 is regularly arranged, crush points 53 are formed in some of the non-joining portions 50 to lower a swelling height, and a space therein is eliminated or narrowed. In other words, a non-joining portion 50 in which a crush point 53 is formed is inhibited from convexly swelling, and is lower in height than another adjacent non-joining portion 50 in which the crush point 53 is not formed. Further, a space is generated between the non-joining portion 50 in which the crush point 53 is formed and the skin of the wearer. In this way, when non-joining portions 50 in which crush points 53 are formed are continuously disposed in a certain direction in a pattern of non-joining portions 50 in which crush points 53 are not formed, a liquid flow path may be formed along the certain direction of connecting the crush points 53. In addition, when the non-joining portion 50 is pressed, the space inside the non-joining portion 50 is narrowed. Thus, diffusion of the liquid may be induced along the certain direction of connecting the crush points 53 due to a capillary phenomenon. Therefore, for example, when a column of the non-joining portions 50 in which the crush points 53 are formed is formed on an outer side of the top sheet 10 in the width direction, and even when urine is excreted around a side edge of the top sheet 10, a diffusion direction of urine may be adjusted to a direction in which side leakage does not occur. In this way, side leakage of urine may be prevented by appropriately controlling the diffusion direction of urine using the crush point 53.

In the top sheet of the invention, the joining portion 40 is preferably formed between adjacent crush points 53. Specifically, when a virtual straight line connecting the crush points 53 is drawn, a part of the joining portion 40 is preferably inevitably positioned between adjacent crush points 53 on the straight line.

As in the above-described configuration, when the joining portion 40 is provided between the crush points 53, a slightly swelling small wall portion 54 is formed between the crush point 53 and the joining portion 40. When the small wall portion 54 is formed in this way, a cushioning property of the upper layer sheet 11 may be maintained when compared to a case in which the upper layer sheet 11 is fully crushed without forming the small wall portion 54. In addition, when the small wall portion 54 is formed, a speed of the liquid flowing along the crush points 53 ranged in one direction may be appropriately decreased. Thus, the liquid may be effectively absorbed around the crush points 53. In other words, when the joining portion 40 is not provided between the crush points 53, there is a possibility that a portion between the crush points 53 may be flat or a groove may be formed therebetween. Then, there is a possibility that the liquid may rapidly flow on the crush points 53, and pass by the crush points 53 without stopping. In this regard, when the small wall portion 54 is provided around the crush point 53 such that the liquid is retained to some extent around the crush point 53 as in the above-described configuration, it is possible to effectively utilize absorption performance of the whole top sheet 10.

The top sheet of the invention is preferably divided into a plurality of joining portion forming regions 70 and a plurality of joining portion non-forming regions 80. The joining portion forming regions 70 are regions in which the joining portion 40 is formed in at least a portion in the width direction. The joining portion non-forming regions 80 are regions in which the joining portion 40 is not formed across the whole width direction between joining portion forming regions 70.

Herein, the plurality of joining portion non-forming regions 80 includes at least a first region 81 whose length in the longitudinal direction (vertical width) corresponds to a first length S₁, and a second region 82 whose length in the longitudinal direction (vertical width) corresponds to a second length S₂. In addition, the second length S₂ is shorter than the first length S₁.

As in the above-described configuration, the joining portion non-forming regions 80 in which the joining portion 40 is not formed across the whole width direction are provided in at least two positions, and vertical widths of the respective joining portion non-forming regions 80 are made different from each other. A joining portion non-forming region 80 having a wide vertical width may smoothly lead urine inside a capillary tube even when the urine is in a state in which salinity concentration and viscosity are high and a movement driving force is small. For this reason, the joining portion non-forming region 80 having the wide vertical width is suitable for diffusion of urine having high salinity concentration. Meanwhile, a joining portion non-forming region 80 having a narrow vertical width may effectively diffuse a liquid in the width direction even when the amount of the liquid is small. Furthermore, the joining portion non-forming region 80 having the narrow vertical width is suitable for diffusion of urine having low salinity concentration. In this way, the top sheet 10 of the invention may appropriately diffuse urine irrespective of whether salinity concentration of urine is high or low. Therefore, the top sheet 10 has a structure that responds to a change in salinity concentration of excreted urine.

A second aspect of the invention relates to an absorbent article 100 including the top sheet 10.

The absorbent article 100 of the invention includes a liquid-permeable top sheet 10, a liquid-impermeable back sheet 20, and an absorber 30 positioned therebetween.

The top sheet 10 is the same as the above-described top sheet according to the first aspect.

In more detail, the top sheet 10 includes an upper layer sheet 11, a lower layer sheet 12, a plurality of joining portions 40 that joins the upper layer sheet 11 and the lower layer sheet 12 to each other, and a plurality of non-joining portions 50 surrounded by the plurality of joining portions 40. Each of the non-joining portions 50 includes a large region 51 having a relatively large area, and a small region 52 having a relatively small area and extending in one direction from the large region 51. The upper layer sheet 11 convexly swells in the large region 51 and the small region 52.

The large region 51 and the small region 52 may hold spaces therein, and insides thereof may be filled with fiber.

### Advantageous Effects of Invention

According to the invention, it is possible to provide a top sheet in which a region easily touching a skin of a wearer has an enhanced quick drying property. The top sheet of the invention maintains a crisp touch for a long time and is excellent in feeling since a region easily touching the skin of the wearer rapidly dries even when the top sheet is touched by a liquid such as urine and damped.

### BRIEF DESCRIPTION OF DRAWINGS

FIG. 1 is a plan view of an absorbent article viewed from a top sheet side.

FIG. 2 is an enlarged view of an embossing pattern formed on a top sheet.

FIG. 3 illustrates components included in the embossing pattern of the top sheet by extracting the components.

FIG. 4 is an explanatory drawing for a scheme of designing a joining portion formed in the top sheet.

FIGS. 5(a) to 5(c) illustrate cross-sectional views of a top sheet according to a first embodiment.

FIG. 6 illustrates an example of a method of manufacturing the top sheet according to the first embodiment.

FIGS. 7(a) to 7(c) illustrate cross-sectional views of a top sheet according to a second embodiment.

FIGS. 8(a) and 8(b) illustrate a cross-sectional view of a region in which a crush point is formed.

FIG. 9 illustrates an example of a method of manufacturing the top sheet according to the second embodiment.

FIG. 10 illustrates an example of a method of manufacturing the region in which the crush point is formed.

FIG. 11 is a plan view for description of a concept of a joining portion forming region and a joining portion non-forming region.

FIG. 12 is an enlarged view of an inside of a dotted frame illustrated in FIG. 11.

FIG. 13 is a cross-sectional view of a region including the joining portion non-forming region.

FIG. 14 illustrates components included in the embossing pattern of the top sheet by extracting the components.

FIG. 15 illustrates a first modified example of the embossing pattern.

FIG. 16 illustrates a second modified example of the embossing pattern.

FIG. 17 illustrates a third modified example of the embossing pattern.

### DESCRIPTION OF EMBODIMENTS

Hereinafter, embodiments for implementing the invention will be described using drawings. The invention is not restricted to a mode described below, and includes a mode appropriately corrected from the mode below within a clear range by those skilled in the art.

In this specification, a “longitudinal direction” basically refers to a direction (Y-axis direction) of connecting a front body part and a rear body part in an absorbent article, and a “width direction” refers to a direction (X-axis direction) two-dimensionally perpendicular to the longitudinal direction.

In addition, in this specification, “A to B” indicates “A or more and B or less”.

### 1. First Embodiment: When Inside of Small Region is Filled with Fiber

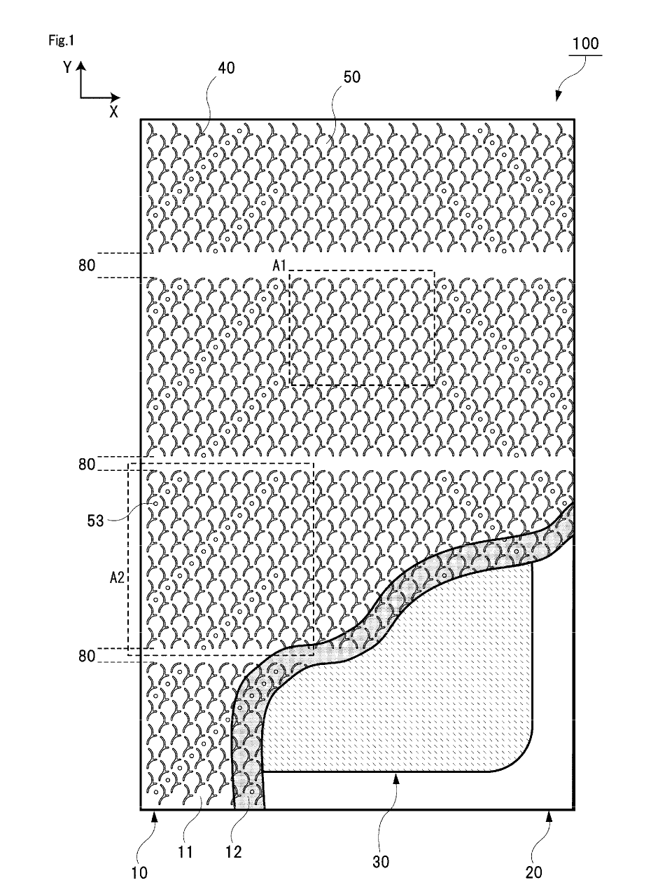

A description will be given of a first embodiment of a disposable diaper according to the invention with reference to FIG. 1 to FIG. 7(c). The first embodiment is a mode in which an inside of a small region 52 of an upper layer sheet 11 described below is filled with fiber.

FIG. 1 is a plan view of an absorbent article 100 according to the first embodiment of the invention viewed from a skin facing surface side. For example, the absorbent article 100 according to the invention may be used for a disposable diaper, a urine pad, a panty liner, a light incontinence pad, and a sanitary napkin. FIG. 1 schematically illustrates an internal structure of the absorbent article 100. As illustrated in FIG. 1, the absorbent article 100 includes a top sheet 10, a back sheet 20, and an absorber 30. The top sheet 10 is a sheet disposed on a skin facing surface side of the absorber 30 to directly come into contact with a skin of a wearer. In addition, the back sheet 20 is disposed on a non-skin facing surface side of the absorber 30. As illustrated in FIG. 1, the top sheet 10 and the back sheet 20 may be joined to each other around the absorber 30. In this way, the absorber 30 is enclosed between the top sheet 10 and the back sheet 20.

The top sheet 10 is a member that directly comes into contact with a skin of a crotch portion of the wearer to allow a liquid such as urine to permeate the absorber 30. For this reason, the top sheet 10 is made of a liquid-permeable material having high flexibility. For example, the liquid-permeable material refers to a material through which 5 ml of room temperature water permeates within one minute when the water is placed thereon under standard atmospheric pressure. Examples of the liquid-permeable material included in the top sheet 10 include woven fabric, nonwoven fabric, or a porous film. In addition, for example, a material obtained by performing a hydrophilic treatment on fiber of a thermoplastic resin such as polypropylene, polyethylene, polyester, or nylon, and then forming a nonwoven fabric using the fiber may be used.

The back sheet 20 is a member for preventing liquid, which permeates the top sheet 10 and is absorbed by the absorber 30, from leaking to the outside. For this reason, the back sheet 20 is made of a liquid-impermeable material. For example, the liquid-impermeable material refers to a material through which 5 ml of room temperature water does not permeate after one minute or more when the water is placed thereon under standard atmospheric pressure. An example of the liquid-impermeable material contained in the back sheet 20 corresponds to a liquid-impermeable film made of polyethylene resin. In particular, it is preferable to use a microporous polyethylene film in which a plurality of minute holes having a size in a range of 0.1 to 4 μm is formed.

The absorber 30 is a member for absorbing liquid such as urine, and holding the absorbed liquid. The absorber 30 is disposed between the liquid-permeable top sheet 10 and the liquid-impermeable back sheet 20. The absorber 30 is made of an absorbent material having a function of absorbing liquid such as urine. A known material may be employed as the absorbent material included in the absorber 30. For example, one type may be independently used or two or more types may be used in combination as the absorbent material among crush pulp (fluff pulp), a high absorbency polymer, or a hydrophilic sheet. Normally, the absorbent material is used by being formed in a shape of a mat having a single layer or a plurality of layers.

The absorbent article 100 has a longitudinal direction and a width direction. In the drawings of this application, the longitudinal direction of the absorbent article 100 is indicated by a Y-axis direction, and the width direction of the absorbent article 100 is indicated by an X-axis direction.

As illustrated in FIG. 1, the top sheet 10 is formed by stacking and bonding a plurality of sheet members together. Two or more sheet members may be included in the top sheet 10. For example, two to five sheet members may be included therein. In the embodiment illustrated in FIG. 1, the top sheet 10 is configured by bonding two sheet members corresponding to an upper layer sheet 11 and a lower layer sheet 12 together. The upper layer sheet 11 is positioned on the skin facing surface side, and the lower layer sheet 12 is positioned on the non-skin facing surface. For this reason, a sheet member directly coming into contact with the skin of the wearer corresponds to the upper layer sheet 11.

Each of the upper layer sheet 11 and the lower layer sheet 12 may be made of a liquid-permeable material such as woven fabric, nonwoven fabric, a porous film, etc. In particular, nonwoven fabric is preferably used as the upper layer sheet 11 and the lower layer sheet 12. Nonwoven fabric made of a known fiber may be used.

Examples of nonwoven fabric may include various nonwoven fabrics such as air-through nonwoven fabric, heat-bond nonwoven fabric, spunbond nonwoven fabric, melt-blown nonwoven fabric, spun lace nonwoven fabric, and needle-punch nonwoven fabric. In addition, when the upper layer sheet 11 and the lower layer sheet 12 are joined to each other using heat fusion, heat-fusible fiber is preferably included in nonwoven fabric. Fiber having a sheath-core structure such as PET/PE, PP/PE, etc. is preferable as heat-fusible fiber. In addition, hydrophilic treatment using a surfactant, etc. is preferably performed on nonwoven fabric.

As illustrated in FIG. 1, the upper layer sheet 11 and the lower layer sheet 12 are joined to each other by embossing. Embossing refers to a processing method of joining two thermoplastic sheet members by interposing the sheet members between an embossing roller provided with convex embossing protrusions having a predetermined pattern on an outer circumferential surface and a plane roller having a flat surface while heating the sheet members. When embossing is performed, a plurality of concave joining portions 40 is formed on the top sheet 10 at positions touched by the embossing protrusions of the embossing roller, and the upper layer sheet 11 and the lower layer sheet 12 are fusion-bonded to each other in the joining portions 40. In addition, in general, when the upper layer sheet 11 and the lower layer sheet 12 are joined to each other by embossing, the upper layer sheet 11 becomes thinner in the joining portions 40. For this reason, the upper layer sheet 11 relatively swells in non-joining portions 50 in which the joining portions 40 are not formed. In this way, the top sheet 10 is formed by heat-fusing the plurality of sheet members together using embossing.

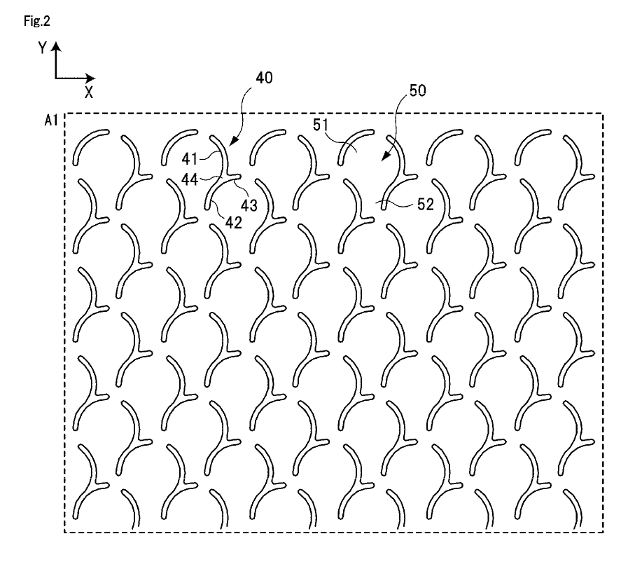

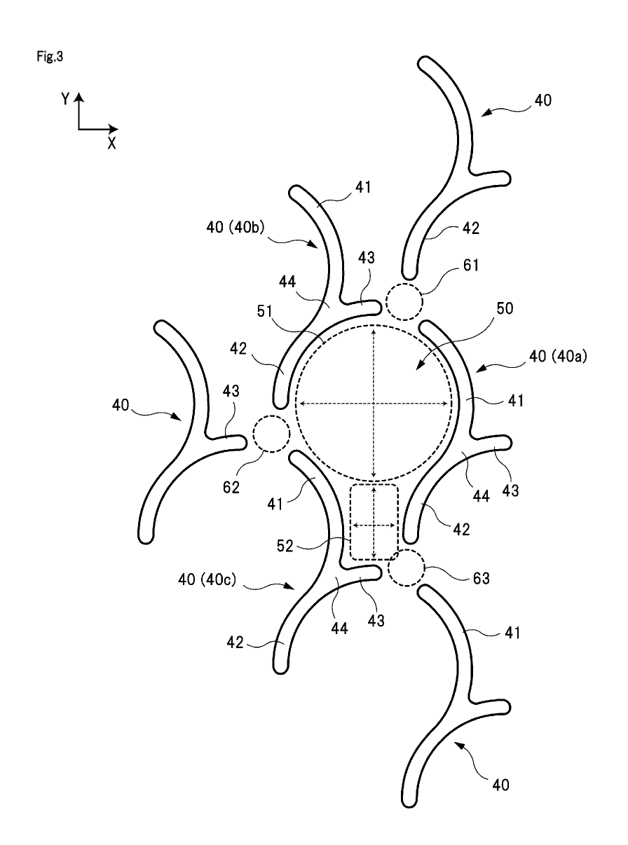

Next, a description will be given of an embossing pattern formed on the top sheet 10. FIG. 2 is an enlarged view of the embossing pattern, and illustrates an inside of a dotted frame illustrated in FIG. 1 by enlarging the inside. In addition, FIG. 3 is another enlarged view illustrating components included in one non-joining portion 50 in the embossing pattern by extracting the components.

As illustrated in FIG. 2 and FIG. 3, the embossing pattern of the top sheet 10 includes a plurality of joining portions 40 that joins the upper layer sheet 11 and the lower layer sheet 12 to each other, and a plurality of non-joining portions 50 surrounded by the plurality of joining portions 40. The plurality of joining portions 40 is regularly disposed at intervals. Specifically, a plurality of joining portions 40 having the same shape, size, and direction is disposed by being arranged in column shapes at certain intervals in the longitudinal direction, and a plurality of columns of the joining portions 40 is formed in the width direction. In addition, in adjacent columns of joining portions 40, the respective joining portions 40 are differently arranged in the longitudinal direction, and a so-called zigzag arrangement is formed. In addition, the respective joining portions 40 are arranged on straight lines in the longitudinal direction and the width direction. In the joining portions 40, the upper layer sheet 11 and the lower layer sheet 12 are heat-fused together. For this reason, the upper layer sheet 11 is concavely hollowed in the joining portions 40. Meanwhile, the non-joining portions 50 correspond to regions surrounded by the plurality of joining portions 40, and the upper layer sheet 11 and the lower layer sheet 12 are not joined to each other in the regions. For this reason, when compared to the joining portions 40, the non-joining portions 50 convexly swells in a direction in which the upper layer sheet 11 touches the skin of the wearer. As illustrated in FIG. 1 to FIG. 3, in the top sheet 10 of the invention, the joining portions 40 and the non-joining portions 50 have novel shapes which have not been present in the past.

As illustrated in FIG. 2 and FIG. 3, a portion of the non-joining portion 50 has a shape extending in a certain direction. For example, the shape of the non-joining portion 50 may be represented by a bulb shape, a keyhole shape, a gourd shape, a pear shape, etc. Specifically, the non-joining portion 50 includes a large region 51 having a relatively large area and a small region 52 having a relatively small area and extending only in one direction from the large region 51. The large region 51 and the small region 52 have no boundary therebetween, and are integrally connected to each other. In other words, the joining portion 40 is not formed between the large region 51 and the small region 52. As illustrated in the figure, the large region 51 and the small region 52 have different shapes. In an example illustrated in the figure, the large region 51 corresponds to a region having a substantially perfect circular shape. In addition, the small region 52 corresponds to a remaining region obtained by excluding the perfect circular large region 51 from the non-joining portion 50. In the example illustrated in the figure, the small region 52 may be regarded as a substantially square region. For this reason, in a preferable mode illustrated in the figure, the non-joining portion 50 assumes a shape obtained by integrally combining the circular large region 51 and the quadrate small region 52 together.

Areas of the large region 51 and the small region 52 are difficult to be accurately measured. However, as illustrated in FIG. 3, when the perfect circular large region 51 is first demarcated, and the rectangular small region 52 is demarcated in a remaining portion within the non-joining portion 50, and widths (widths in the X-axis direction) thereof are compared, the width of the large region 51 is clearly wider than the width of the small region. Similarly, when the perfect circular large region 51 is first demarcated, and the rectangular small region 52 is demarcated in a remaining portion within the non-joining portion 50, and lengths (lengths in the Y-axis direction) thereof are compared, the length of the large region 51 is longer than the length of the small region. In this way, the large region 51 having the larger area may be regarded as a region having a larger width and length than those of the small region 52. Similarly, the small region 52 having the small area may be regarded as a region having a smaller width and length than those of the large region 51. The width and length of the small region 52 are preferably half (½) or less the width and length of the large region 51, respectively.

In addition, as illustrated in FIG. 2, the plurality of non-joining portions 50 having substantially the same shapes is regularly formed in the embossing pattern of the top sheet 10. Further, directions in which small regions 52 extend from large regions 51 are identical to one another in the respective non-joining portions 50. For this reason, a unified regular pattern is represented on a surface of the top sheet 10, and the pattern is beautiful in terms of appearance.

The above-described non-joining portion 50 having a particular shape is formed by a plurality of joining portions 40 having a particular shape. As illustrated in FIG. 1 to FIG. 3, in the invention, the joining portion 40 preferably has a shape in which three line portions 41 to 43 branch off in three different directions. In the example illustrated in FIG. 1 to FIG. 3, each of the joining portions 40 has a shape of “λ”. In addition, the embossing pattern of the top sheet 10 may be formed by the joining portions 40 having the same shape (λ shape).

A detailed description will be given of a shape and arrangement of the joining portions 40 with reference to FIG. 3. As illustrated in FIG. 3, the joining portion 40 has a shape in which the first line portion 41, the second line portion 42, and the third line portion 43 branch off from a junction 44 thereof in different directions. In other words, proximal ends (one ends) of the first line portion 41, the second line portion 42, and the third line portion 43 are connected at the junction 44, and distal ends (the other ends) of the first line portion 41, the second line portion 42, and the third line portion 43 are directed in different directions. In addition, at least the first line portion 41 and the second line portion 42 preferably have a shape curved in a circular arc shape, or a shape bent at one or more turning points. In the example illustrated in FIG. 3, the first line portion 41 and the second line portion 42 are curved in circular arc shapes. In addition, as illustrated in FIG. 3, in the first line portion 41 and the second line portion 42 having the circular arc shapes, directions in which arcs bulge out (directions in which the arcs warp) preferably correspond to opposite directions. In other words, when the first line portion 41 and the second line portion 42 are connected to each other, a soft S-shape is formed. In addition, the third line portion 43 may have a circular arc shape similarly to the first line portion 41 and the second line portion 42, or have a linear shape. In addition, directions in which the first line portion 41 and the second line portion 42 extend are preferably substantially orthogonal to a direction in which the third line portion 43 extends. For example, in the example illustrated in FIG. 1 to FIG. 3, while the first line portion 41 and the second line portion 42 substantially extend in the longitudinal direction (Y-axis direction), the third line portion 43 substantially extends in the width direction (X-axis direction). The directions in which the first line portion 41 and the second line portion 42 extend substantially correspond to opposite directions. For example, while the first line portion 41 extends in a positive direction on a Y axis (upward in the figure), the second line portion 42 extends in a negative direction on the Y axis (downward in the figure). In this sense, the directions in which the first line portion 41 and the second line portion 42 extend are substantially orthogonal to the direction in which the third line portion 43 extends. The first line portion 41 and the second line portion 42 may extend in the width direction (X-axis direction), and the third line portion 43 may extend in the longitudinal direction (Y-axis direction).

In addition, as illustrated in FIG. 3, the first line portion 41, the second line portion 42, and the third line portion 43 may have different lengths. In the example illustrated in FIG. 3, the first line portion 41 is longer than the second line portion 42, and the second line portion 42 is longer than the third line portion 43 (the first line portion 41>the second line portion 42>the third line portion 43). In addition, when all the respective line portions 41 to 43 are formed in circular arc shapes, curvatures (or radii of curvatures) of the respective line portions 41 to 43 may be different from one another or identical to one another. In the example illustrated in the figure, all the curvatures of the respective line portions 41 to 43 correspond to the same value.

As illustrated in FIG. 3, three joining portions 40 are positioned around one certain non-joining portion 50. The three joining portions 40 positioned around the one certain non-joining portion 50 are defined as a first joining portion 40a, a second joining portion 40b, and a third joining portion 40c. In this case, a portion around the one certain non-joining portion 50 is demarcated by elements (i) to (vi) below.

(i) First line portion 41 and second line portion 42 of first joining portion 40a

(ii) Second line portion 42 and third line portion 43 of second joining portion 40b

(iii) Third line portion 43 and first line portion 41 of third joining portion 40c

(iv) First gap 61 between first line portion 41 of first joining portion 40a and third line portion 43 of second joining portion 40b

(v) Second gap 62 between second line portion 42 of second joining portion 40b and first line portion 41 of third joining portion 40c

(vi) Third gap 63 between third line portion 43 of third joining portion 40c and second line portion 42 of first joining portion 40a

In other words, as illustrated in FIG. 3, firstly, the joining portions 40 are formed such that a distal end of the first line portion 41 of the first joining portion 40a and a distal end of the third line portion 43 of the second joining portion 40b butt to each other (approach each other). In this instance, the first gap 61 is formed. In addition, the joining portions 40 are formed such that a distal end of the second line portion 42 of the second joining portion 40b and a distal end of the first line portion 41 of the third joining portion 40c butt to each other (approach each other). In this instance, the second gap 62 is formed. Further, the joining portions 40 are formed such that a distal end of the third line portion 43 of the third joining portion 40c and a distal end of the second line portion 42 of the first joining portion 40a butt to each other (approach each other). In this instance, the third gap 63 is formed. The first gap 61, the second gap 62, and the third gap 63 may correspond to a distance in a range of about 0.1 mm to 20 mm. According to such a principle, a plurality of joining portions 40 is disposed, and a regular embossing pattern is formed.

As illustrated in FIG. 3, the circular large region 51 and the quadrate small region 52 are formed in the non-joining portion 50, a portion around which is demarcated by the above elements (i) to (vi). The portion around the circular large region 51 is demarcated by the first line portion 41 of the first joining portion 40a, the second line portion 42/the third line portion 43 of the second joining portion 40b, the first gap 61, and the second gap 62. Herein, the first line portion 41 of the first joining portion 40a and the second line portion 42/the third line portion 43 of the second joining portion 40b are curved to bulge out toward an outside of the non-joining portion 50 (the large region 51). For this reason, the large region 51 has a substantially circular shape, and the area thereof becomes larger. Meanwhile, a portion around the quadrate small region 52 is demarcated by the second line portion 42 of the first joining portion 40a, the first line portion 41/the third line portion 43 of the third joining portion 40c, and the third gap 63. Herein, the second line portion 42 of the first joining portion 40a and the first line portion 41/the third line portion 43 of the third joining portion 40c are curved to bulge out toward an inside of the non-joining portion 50 (the small region 52). For this reason, the area of the small region 52 becomes smaller.

In addition, as illustrated in FIG. 2 and FIG. 3, it is considered that each of the plurality of non-joining portions 50 corresponds to an independent region divided by the joining portions 40, and is not connected to another non-joining portion 50. However, in the strict sense, it can be understood that non-joining portions 50 are connected to each other through the gaps 61 to 63. However, since the gaps 61 to 63 correspond to a region formed when distal ends of line portions of three joining portions 40 butt to one another as illustrated in FIG. 3, the gaps 61 to 63 are concavely hollowed at a similar level to that of the joining portion 40 when the upper layer sheet 11 and the lower layer sheet 12 are actually joined to each other. At least the gaps 61 to 63 do not convexly swell. Specifically, each of the gaps 61 to 63 positioned around the one certain non-joining portion 50 corresponds to a region in which the first line portion 41, the second line portion 42, and the third line portion 43 of three joining portions 40 butt to one another. For this reason, as a result of forming the three joining portions 40, the gaps 61 to 63 crush together with the three joining portions 40 or are inhibited from swelling. Therefore, adjacent convex non-joining portions 50 are not connected to each other through the gaps 61 to 63, and the respective convex non-joining portions 50 are substantially independent of each other. In addition, as illustrated in FIG. 2 and FIG. 3, the third line portion 43 of each of the joining portions 40 separates non-joining portions 50 adjacent to each other in the longitudinal direction (Y direction) such that the non-joining portions 50 are not connected to each other. In this regard, the third line portion 43 of each of the joining portions 40 has significance.

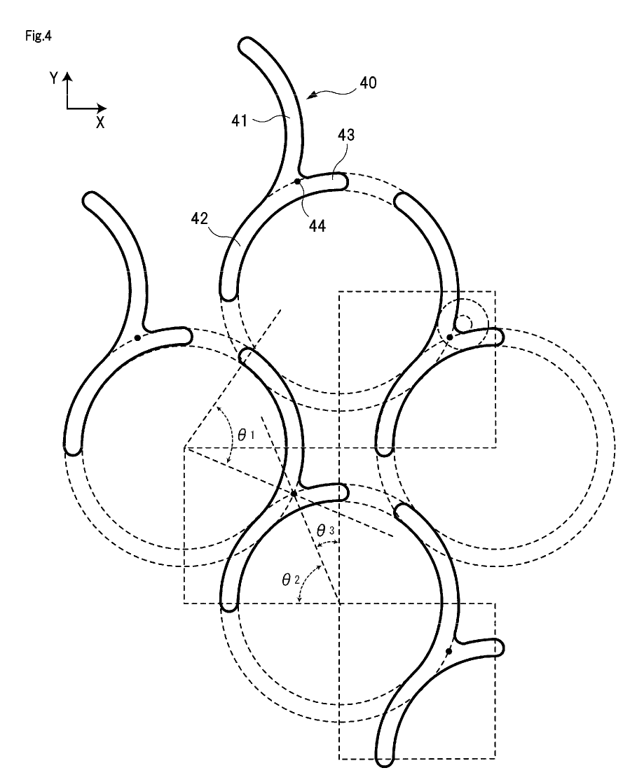

Next, a description will be given of an example of a method of designing the embossing pattern with reference to FIG. 4. As illustrated in FIG. 4, first, a latticed pattern is presumed in which a plurality of virtual perfect circles having a certain amount of width is disposed to come into contact with one another such that width portions thereof overlap each other. In other words, the latticed pattern refers to a pattern in which a certain virtual perfect circle is disposed to come into contact with other virtual perfect circles at an interval of 90 degrees. All the plurality of virtual perfect circles is formed to have the same radius (r). The radius (r) of the virtual circle is preferably in a range of 1.0 mm to 11.0 mm, and more preferably in a range of 2.0 mm to 5.0 mm. A width of the virtual perfect circle is identical to a width of the joining portion 40.

In this case, firstly, the first line portion 41 of the joining portion 40 is formed in a circular arc shape curved along a circumference of a certain virtual perfect circle. A length L₁ of the first line portion 41 is obtained using [Equation] L₁=2πr×(θ₁/360). Herein, “r” denotes a radius of the virtual perfect circle. In addition, when each of the distal end and the proximal end of the first line portion 41 is connected to a center of the virtual perfect circle by straight lines, “θ₁” denotes an angle formed by the respective straight lines (that is, an angle of a sector). For example, θ₁ may be set to a range of 60 degrees to 100 degrees, 70 degrees to 90 degrees, or 75 degrees to 85 degrees.

In addition, the second line portion 42 and the third line portion 43 of the joining portion 40 are formed along a circumference of a different virtual perfect circle from that of the first line portion 41 described above. That is, the second line portion 42 and the third line portion 43 are positioned on a circumference of a virtual perfect circle adjacent to the virtual perfect circle on which the first line portion 41 is formed. For this reason, the junction 44 of the respective line portions 41 to 43 is positioned on a boundary between two adjacent virtual perfect circles. In addition, the second line portion 42 and the third line portion 43 are positioned on a circumference of the same virtual perfect circle. Therefore, the second line portion 42 and the third line portion 43 integrally form one circular arc shape. For this reason, it may be considered that each of the joining portions 40 has a shape obtained by combining two circular arcs corresponding to a circular arc formed by the first line portion 41 and a circular arc formed by the second line portion 42 and the third line portion 43.

A length L₂ of the second line portion 42 is obtained using [Equation] L₂=2πr×(θ₂/360). When each of the distal end and the proximal end of the second line portion 42 is connected to a center of the virtual perfect circle by straight lines, “θ₂” denotes an angle formed by the respective straight lines (that is, an angle of a sector). For example, θ₂ may be set to a range of 45 degrees to 85 degrees, 50 degrees to 80 degrees, or 55 degrees to 70 degrees. In addition, a length L₃ of the third line portion 43 is obtained using [Equation] L₃=2πr×(θ₃/360). When each of the distal end and the proximal end of the third line portion 43 is connected to a center of the virtual perfect circle by straight lines, “θ₃” denotes an angle formed by the respective straight lines (that is, an angle of a sector). For example, θ₂ may be set to a range of 5 degrees to 45 degrees, 10 degrees to 40 degrees, or 20 degrees to 35 degrees. In addition, as illustrated in FIG. 4, a sum of θ₂ and θ₃ is preferably 90 degrees (±5 degrees).

In this way, it is possible to relatively easily design the joining portions 40 having the particular shape. In addition, the respective joining portions 40 are disposed based on a pattern of virtual perfect circles disposed in a lattice shape, and thus the respective joining portions 40 may be regularly disposed. The distal ends of the respective line portions 41 and 43 are preferably formed as smooth curves through rounding. In addition, an acute angle is formed at a connection position of the first line portion 41 and the third line portion 43, and thus the connection position is preferably subjected to rounding to form a curve.

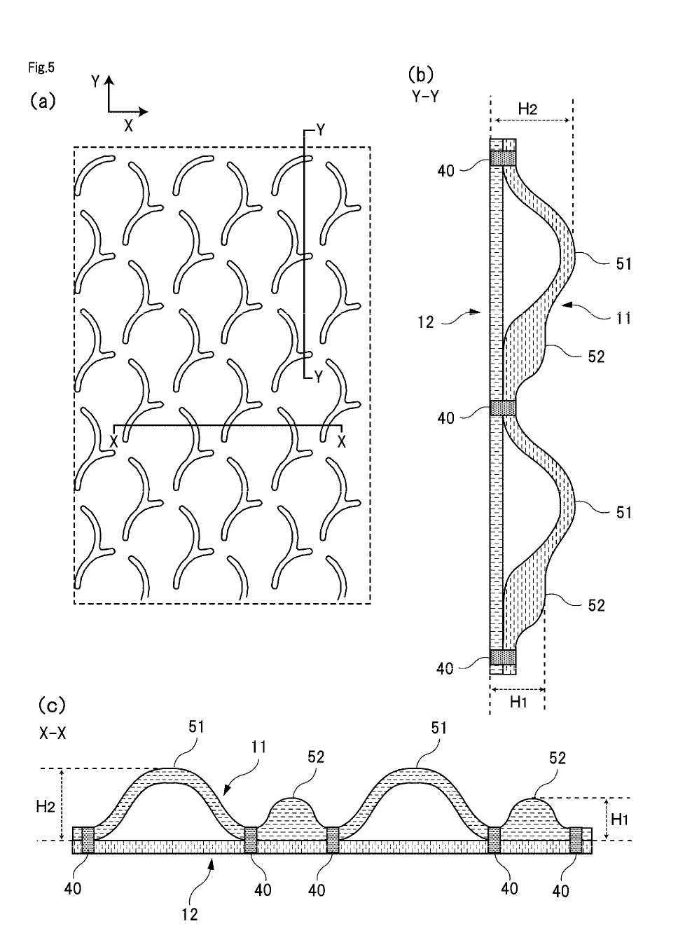

FIGS. 5(a) to 5(c) schematically illustrate cross sections of the top sheet 10. FIG. 5(b) is a cross-section view taken along Y-Y line illustrated in FIG. 5(a), and FIG. 5(c) is a cross-section view taken along X-X line illustrated in FIG. 5(a). As illustrated in FIG. 5(b) and FIG. 5(c), each of the non-joining portions 50 surrounded by a plurality of joining portions 40 includes one large region 51 and one small region 52. In addition, the upper layer sheet 11 convexly swells in the large region 51 and the small region 52. Further, a height at which the upper layer sheet 11 swells in the large region 51 is higher than a height at which the upper layer sheet 11 swells in the small region 52. For example, when a height H₁ of the small region 52 is set to 100%, a height H₂ of the large region 51 is preferably in a range of 120% to 300%, 130% to 250%, or 140% to 200%.

In addition, in an example illustrated in FIG. 5(b) and FIG. 5(c), the large region 51 of the non-joining portion 50 is formed in a dome shape that holds a space on the inside. In other words, the upper layer sheet 11 is separated from the lower layer sheet 12 and convexly swells in the large region 51. For this reason, a space is formed between the upper layer sheet 11 and the lower layer sheet 12. Meanwhile, an inside of the small region 52 of the non-joining portion 50 is filled with fiber included in the upper layer sheet 11. For this reason, in the small region 52, fiber of the upper layer sheet 11 at least partially comes into contact with the lower layer sheet 12. In this way, a density of fiber included in the upper layer sheet 11 may be low in the large region 51 and high in the small region 52.

In order to form the large region 51 in the dome shape, the upper layer sheet 11 may be pressed from the non-skin facing surface side (rear surface side) to convexly swell the upper layer sheet 11 in a region corresponding to the large region 51 before the upper layer sheet 11 and the lower layer sheet 12 are overlapped with each other. Thereafter, when the upper layer sheet 11 and the lower layer sheet 12 are overlapped with each other to join both sheets in the respective joining portions 40, a space is formed between the upper layer sheet 11 and the lower layer sheet 12 in the large region 51. In addition, when the upper layer sheet 11 is pressed in the region corresponding to the large region 51, fiber included in the upper layer sheet 11 may be allowed to flow from the large region 51 to the small region 52. In this way, a fiber density of the large region 51 may be decreased, and a fiber density of the small region 52 may be increased. In this way, it is possible to form the large region 51 which is high in height and low in fiber density, and the small region 52 which is low in height and high in fiber density.

As described in the foregoing, when a liquid touches the top sheet 10 at the time of urination of the wearer, etc., the liquid may be rapidly moved from the large region 51 to the small region 52 by adjusting the heights and the fiber densities of the large region 51 and the small region 52. That is, the liquid flows down from the large region 51 high in height to the small region 52 low in height. In addition, the liquid is absorbed by the small region 52 high in fiber density rather than the large region 51 low in fiber density. For this reason, even when the sheet is damp due to the liquid touching the sheet, the large region 51 rapidly dries. The large region 51 has a large area and a high height, and thus easily touches the skin of the wearer. Therefore, when a drying time of the large region 51 is shortened, it is possible to shorten a time at which the liquid touches the skin of the wearer, and to prevent the wearer from feeling an unpleasant feeling. Meanwhile, the liquid moved from the large region 51 is temporarily retained in the small region 52. For this reason, the small region 52 takes a longer time to fully dry when compared to the large region 51. However, the small region 52 has a small area and a low height, and thus rarely touches the skin of the wearer. For this reason, even when the small region 52 takes time to dry, the wearer may rarely feel an unpleasant feeling. In addition, in the top sheet 10 of the invention, the small region 52 is provided at only one location for one large region 51. For this reason, a liquid coming into contact with the large region 51 moves only in one direction toward the small region 52 protruding from the large region 51. In other words, in the top sheet 10 of the invention, the liquid does not diffuse in every direction around the large region 51, and thus the number of regions in which the liquid is temporarily retained does not excessively increase. When a plurality of (two or more) regions in which the liquid is retained is present around the large region 51, there is a demerit that the skin of the wearer is more likely to touch the liquid retaining regions. In addition, there is a concern that the number of liquid retaining regions touched by the skin of the wearer may increase. For this reason, the wearer may feel an unpleasant feeling. On the other hand, when the small region 52 for retaining the liquid is provided at only one location for one large region 51 as in the invention, the number of liquid retaining regions may be suppressed to the minimum. Therefore, according to the invention, a temporary liquid retaining region may be appropriately controlled not to give an unpleasant feeling to the wearer.

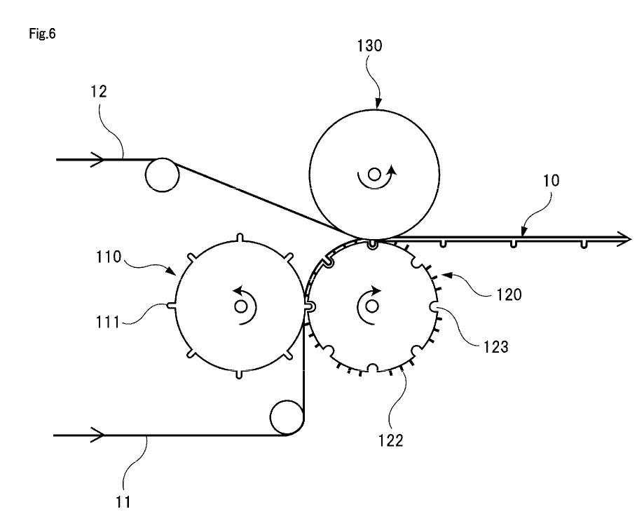

Next, a description will be given of a method of manufacturing the top sheet 10 described above with reference to FIG. 6. FIG. 6 illustrates a method of forming a convex swelling portion on the upper layer sheet 11 in the upper layer sheet 11 and the lower layer sheet 12 included in the top sheet 10, and then joining the upper layer sheet 11 and the lower layer sheet 12 to each other in a predetermined embossing pattern. As illustrated in FIG. 6, an apparatus for manufacturing the top sheet 10 includes a pin roller 110, an embossing roller 120, and a plane roller 130.

As illustrated in FIG. 6, the pin roller 110 has a projection portion 111 that convexly projects at a plurality of locations on a peripheral surface thereof. The projection portion 111 of the pin roller 110 is provided to form the large region 51 that convexly swells on the upper layer sheet 11 of the top sheet 10. For this reason, the projection portion 111 is provided at a position that comes into contact with a region scheduled to become the large region 51 of the upper layer sheet 11.

A plurality of convex embossing protrusions 122 disposed in a predetermined pattern and a plurality of concave hollow portions 123 are formed on a peripheral surface of the embossing roller 120. The embossing protrusions 122 are provided to join the upper layer sheet 11 and the lower layer sheet 12 of the top sheet 10 in a predetermined embossing pattern. The embossing protrusions 122 of the embossing roller 120 may be heated by a heating device (not illustrated). In addition, the hollow portions 123 are provided at positions corresponding to a plurality of projection portions 111 provided on the peripheral surface of the pin roller 110, and have shapes capable of receiving the projection portions 111. The embossing protrusions 122 of the embossing roller 120 are not formed at positions at which the hollow portions 123 are provided.

A peripheral surface of the plane roller 130 corresponds to a smooth surface. The plane roller 130 is provided to interpose the upper layer sheet 11 and the lower layer sheet 12 of the top sheet 10 between the embossing protrusions 122 of the embossing roller 120 and the plane roller 130 to press and heat the sheets, thereby heat-fusing both sheets. The peripheral surface of the plane roller 130 may be made of metal or rubber. In addition, the peripheral surface of the plane roller 130 may be heated by the heating device (not illustrated).

As illustrated in FIG. 6, the pin roller 110 and the embossing roller 120 are disposed to face each other, and the upper layer sheet 11 is introduced between both rollers. In addition, the embossing roller 120 and the plane roller 130 are disposed to face each other, and the upper layer sheet 11 and the lower layer sheet 12 are introduced in an overlapping state between both rollers.

As illustrated in FIG. 6, the upper layer sheet 11 drawn from an original fabric roll (not illustrated) is introduced between the pin roller 110 and the embossing roller 120 via one or a plurality of guide rollers. Referring to the upper layer sheet 11, the non-skin facing surface (surface not directly coming into contact with the skin of the wearer) comes into contact with the pin roller 110, and the skin facing surface (surface directly coming into contact with the skin of the wearer) comes into contact with the embossing roller 120. In this instance, when the upper layer sheet 11 is fit to the hollow portions 123 of the embossing roller 120 while being pressed against the projection portions 111 of the pin roller 110, pressed portions swell. In this way, a portion swelling in a dome shape toward the skin facing surface side (the large region 51 of the non-joining portion 50) is formed on the upper layer sheet 11. Thereafter, the upper layer sheet 11 is introduced between the embossing roller 120 and the plane roller 130 while coming into contact with the peripheral surface of the embossing roller 120.

Meanwhile, the lower layer sheet 12 drawn from another original fabric roll (not illustrated) is introduced between the embossing roller 120 and the plane roller 130 via one or a plurality of guide rollers. The upper layer sheet 11 and the lower layer sheet 12 overlap each other between the embossing roller 120 and the plane roller 130. In this instance, the skin facing surface side of the upper layer sheet 11 comes into contact with the embossing roller 120, and the non-skin facing surface side of the lower layer sheet 12 comes into contact with the plane roller 130. The upper layer sheet 11 and the lower layer sheet 12 are interposed between the embossing roller 120 and the plane roller 130 in a stacked state, and heat-fused by being heated and pressed. The upper layer sheet 11 and the lower layer sheet 12 are heat-fused according to the embossing pattern of the plurality of embossing protrusions 122 formed on the peripheral surface of the embossing roller 120. In this way, a plurality of concave joining portions 40 hollowed toward the non-skin facing surface side is formed on the top sheet 10. In the upper layer sheet 11 and the lower layer sheet 12, regions coming into contact with the embossing protrusions 122 of the embossing roller 120 correspond to the joining portions 40, and a region surrounded by a plurality of joining portions 40 corresponds to the non-joining portion 50.

In this way, when the upper layer sheet 11 is pressed by the pin roller 110, and then the upper layer sheet 11 and the lower layer sheet 12 are joined by the embossing roller 120, the large region 51 of the non-joining portion 50 may be formed in a dome shape that holds a space therein. In addition, when the upper layer sheet 11 is pressed by the projection portions 111 of the pin roller 110, fiber included in the upper layer sheet 11 may be allowed to flow from the large region 51 to the small region 52 of the non-joining portion 50. For this reason, it is possible to decrease the fiber density of the large region 51, and improve the fiber density of the small region 52 at the same time.

### 2. Second Embodiment: When Small Region Holds Space Therein

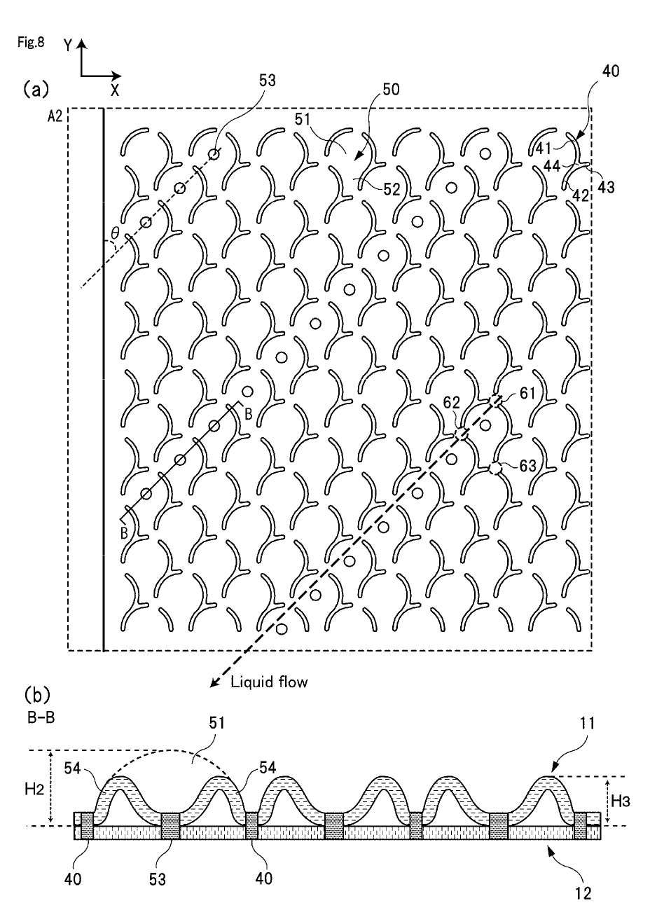

Next, a description will be given of a second embodiment of the disposable diaper according to the invention with reference to FIG. 8(a) to FIG. 10. The second embodiment corresponds to a mode in which the small region 52 of the upper layer sheet 11 holds a space therein. In description of the second embodiment, a similar configuration to that of the above-described first embodiment will not be described, and a difference from the first embodiment will be described in detail.

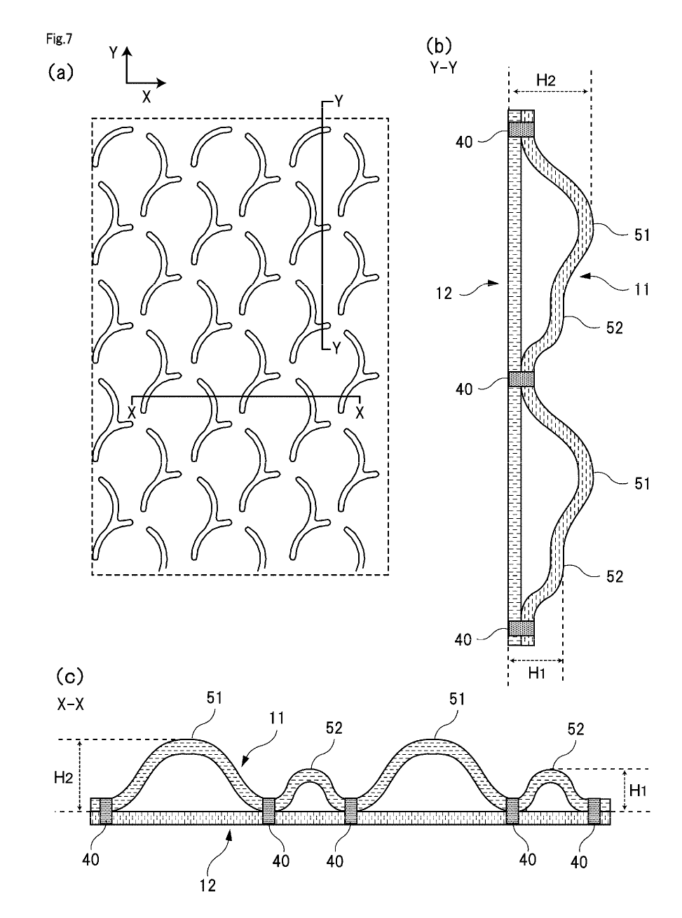

FIGS. 7(a) to 7(c) illustrate cross-sectional views of a top sheet according to the second embodiment, and correspond to FIGS. 5(a) to 5(c). FIG. 7(b) is a cross-section view taken along Y-Y line illustrated in FIG. 7(a), and FIG. 7(c) is a cross-section view taken along X-X line illustrated in FIG. 7(a). As illustrated in FIGS. 7(b) and 7(c), the large region 51 and the small region 52 of the non-joining portion 50 are formed in dome shapes that hold spaces therein. In other words, the upper layer sheet 11 is separated from the lower layer sheet 12 to convexly swell in the large region 51 and the small region 52. For this reason, the spaces are formed between the upper layer sheet 11 and the lower layer sheet 12. These spaces are not filled with fiber included in the upper layer sheet 11 or the lower layer sheet 12.

In order to form the large region 51 and the small region 52 in the dome shapes, the upper layer sheet 11 may be pressed from the non-skin facing surface side (rear surface side) to convexly swell the upper layer sheet 11 in regions corresponding to the large region 51 and the small region 52 before the upper layer sheet 11 and the lower layer sheet 12 are overlapped with each other. Alternatively, the upper layer sheet 11 may be sucked from the skin facing surface side (front surface side) to convexly swell the upper layer sheet 11 in the regions corresponding to the large region 51 and the small region 52. In addition, thereafter, when the upper layer sheet 11 and the lower layer sheet 12 are overlapped with each other to join both sheets in the respective joining portions 40, spaces are formed between the upper layer sheet 11 and the lower layer sheet 12 in the large region 51 and the small region 52. In this way, as illustrated in FIGS. 7(a) to 7(c), the upper layer sheet 11 may be separated from the lower layer sheet 12 to form a state in which spaces are held inside the large region 51 and the small region 52. In addition, in the present embodiment, the large region 51 swells higher than the small region 52. Accordingly, the space formed in the large region 51 is wider than the space formed in the small region 52. For example, when the large region 51 and the small region 52 are formed, adjustment may be performed such that the large region 51 is largely swelled, and the small region 52 is slightly swelled by adjusting a height at which the upper layer sheet 11 is pressed from the non-skin facing surface side. Alternatively, the large region 51 may be largely swelled, and the small region 52 may be slightly swelled by adjusting a suction force for sucking the upper layer sheet 11 from the skin facing surface side.

When the heights of the large region 51 and the small region 52 are different from each other, and the spaces are formed inside both regions as described above, and when a liquid comes into contact with the top sheet 10, the liquid may be rapidly moved from the large region 51 to the small region 52. In other words, the liquid flows down from the large region 51 high in height to the small region 52 low in height. In addition, the liquid is attracted to the small region 52 having a narrow space rather than the large region 51 having a wide space. For this reason, even when the sheet is damp by touching the liquid, an apex of the large region 51 rapidly dries. The large region 51 has a large area and a high height, and thus is easily touched by the skin of the wearer. Therefore, when a drying time at the apex of the large region 51 is shortened, it is possible to shorten a time at which the liquid touches the skin of the wearer, and to prevent the wearer from feeling an unpleasant feeling. Meanwhile, the liquid moved from the large region 51 is temporarily retained in the small region 52. For this reason, the small region 52 may take a longer time to fully dry when compared to the large region 51. However, the small region 52 has a small area and a low height, and thus rarely touched by the skin of the wearer. For this reason, even when the small region 52 takes time to dry, the wearer may rarely feel an unpleasant feeling. In addition, when the space is formed in the small region 52, a drying time of the small region 52 may be shortened.

In addition, as illustrated in FIGS. 7(a) to 7(c), when each of the large region 51 and the small region 52 have the dome shapes that hold the spaces, repulsive forces of the large region 51 and the small region 52 with respect to pressing may correspond to the same level. In more detail, when the spaces are formed in both the large region 51 and the small region 52, and the repulsive forces of both regions are set to the same level or maintained at moderately different levels as in the present embodiment, a touch of the whole top sheet 10 may become softer.

FIG. 8(a) is an enlarged view of the embossing pattern, and illustrates an inside of a dotted frame indicated by a symbol A2 in FIG. 1 by enlarging the inside. As illustrated in FIG. 1, FIG. 8(a) illustrates an outer region of the top sheet 10 in the width direction by enlarging the region. In addition, FIG. 8(b) is a cross-sectional view taken along B-B line illustrated in FIG. 8(a). Herein, a description will be given of an example of a case in which a crush point 53 is formed in the top sheet with reference to FIGS. 8(a) and 8(b).

In the example illustrated in FIGS. 8(a) and 8(b), a plurality of convex non-joining portions 50 is formed by performing embossing on the top sheet, crush points 53 are formed in some of the non-joining portions 50, and the non-joining portions 50 in which the crush points 53 are formed are arranged in a certain direction. In this way, a flow path for a liquid is formed along the certain direction in which the crush points 53 are connected to each other. In addition, when a protrusion of the non-joining portion 50 is pressed, the space inside the non-joining portion is narrowed. Thus, a diffusion of the liquid may be induced along the certain direction in which the crush points 53 are formed due to a capillary phenomenon. Therefore, it is possible to control a diffusion direction of the liquid flowing on the surface of the top sheet.

As illustrated in FIG. 8(a), the plurality of crush points 53 is formed in the outer region of the top sheet 10 in the width direction. The crush points 53 are formed in some of the plurality of non-joining portions 50 formed on the top sheet 10. Specifically, the crush point 53 is formed in the large region 51 of the non-joining portion 50. As illustrated in FIG. 8(b), the crush point 53 is formed by pressing the large region 51 of the non-joining portion 50 in a thickness direction. For this reason, a swelling height of the large region 51 in which the crush point 53 is formed lower. At the same time, the space inside the large region 51 narrows or disappears. The crush point 53 may be formed only by pressing the upper layer sheet 11 or formed by joining the upper layer sheet 11 and the lower layer sheet 12 together in the large region 51. At least, at the crush point 53, the upper layer sheet 11 and the lower layer sheet 12 preferably touch each other. In particular, the upper layer sheet 11 and the lower layer sheet 12 are preferably joined together at the crush point 53 in order to maintain the crush point 53. The upper layer sheet 11 and the lower layer sheet 12 may be heat-fused by heat sealing, ultrasonic sealing, etc.

In addition, as illustrated in FIG. 8(a), the non-joining portions 50 in which the crush points 53 are formed are continuously formed along the certain direction. In other words, the non-joining portions 50 in which the crush points 53 are formed are adjacent to each other in the certain direction. In the present embodiment, a direction in which the non-joining portions 50 in which the crush points 53 are formed are continued is a direction other than the longitudinal direction (Y-axis direction) and the width direction (X-axis direction) of the absorbent article. Specifically, the direction is a direction inclined at a predetermined angle with respect to the longitudinal direction and the width direction. For example, as illustrated in FIG. 8(a), when the continuously formed crush points 53 are connected by a virtual straight line, an angle (θ) at which the straight line is inclined with respect to the longitudinal direction (Y-axis direction) may be set to 15 degrees to 75 degrees, 30 degrees to 60 degrees, 40 degrees to 50 degrees, or 45 degrees.

As illustrated in FIG. 8(a), the non-joining portions 50 in which the crush points 53 are formed are disposed in a pattern of non-joining portions 50 in which the crush point 53 is not formed. In other words, at least two non-joining portions 50 in which the crush point 53 is not formed are present around a non-joining portion 50 in which the crush point 53 is formed. Specifically, in the embossing pattern illustrated in FIG. 8(a), six non-joining portions 50 are present around one non-joining portion 50 by being adjacent thereto. Herein, in observation around the non-joining portion 50 in which the crush point 53 is formed, one or two non-joining portions 50 in which crush points 53 are formed are present around the non-joining portion 50, and the crush point 53 is not formed in five or four remaining non-joining portions 50. In this way, at least two non-joining portions 50 in which the crush point 53 is not formed are preferably present around the non-joining portion 50 in which the crush point 53 is formed by being adjacent thereto.

In this way, when the crush points 53 are formed in some of the convexly swelling non-joining portions 50, and the non-joining portions 50 in which the crush points 53 are formed are continuously disposed along one direction, a liquid flow path may be formed along a direction in which the crush points 53 are continued. In addition, when the non-joining portion 50 is pressed, the space inside the non-joining portion 50 is narrowed. Thus, the liquid is induced along the certain direction in which the crush points 53 are connected due to the capillary phenomenon. In this way, the liquid such as urine may be guided along the direction in which the crush points 53 are continued. Therefore, when a column of the non-joining portions 50 in which the crush points 53 are formed is formed on an outer side of the top sheet 10 in the width direction, and even when urine is excreted around a side edge of the top sheet 10, a diffusion direction of urine may be adjusted to a direction in which side leakage does not occur. In this way, side leakage of urine may be prevented by appropriately controlling the diffusion direction of urine using the crush point 53.

In addition, as illustrated in FIG. 8(b), the joining portion 40 is preferably formed between adjacent crush points 53. Specifically, when a virtual straight line connecting the crush points 53 is drawn, a part of the joining portion 40 is inevitably positioned between crush points 53 on the straight line. A region in which the joining portion 40 is not formed is not present between the adjacent crush points 53. For this reason, in a cross-sectional view taken along the straight line that connects the crush points 53, the crush points 53 and the joining portions 40 are alternately formed as illustrated in FIG. 8(b). In this case, a small wall portion 54 in which the upper layer sheet 11 slightly swells is formed between the crush point 53 and the joining portion 40. A space may be held inside the small wall portion 54. A swelling height (H₃) of the small wall portion 54 is lower than a swelling height (H₂) of the large region 51 of the non-joining portion 50 in which the crush point 53 is not formed. For example, the swelling height (H₃) of the small wall portion 54 is preferably in a range of 20% to 80% and particularly preferably in a range of 30% to 70% with respect to the swelling height (H₂) of the normal large region 51.

When the small wall portion 54 is formed in this way, a cushioning property of the upper layer sheet 11 may be maintained when compared to a case in which the upper layer sheet 11 is fully crushed without forming the small wall portion 54. In addition, when the small wall portion 54 is formed, a speed of the liquid flowing along the crush points 53 ranged in one direction may be decreased. Thus, the liquid may be effectively absorbed around the crush points 53. In other words, when the joining portion 40 is not provided between the crush points 53, there is a possibility that a portion between the crush points 53 may be flat or a groove may be formed therebetween. Then, there is a possibility that the liquid may rapidly flow on the crush points 53, and pass by the crush points 53 without stopping. In this regard, the small wall portion 54 is preferably provided around the crush point 53 as described above such that the liquid is retained to some extent around the crush point 53, thereby effectively utilizing absorption performance of the whole top sheet 10.

In addition, in FIG. 8(a), a direction in which the liquid flows is indicated by a dotted line arrow. Here, the liquid flows along the direction in which the non-joining portions 50, in which the crush points 53 are formed, are continued. As indicated by this arrow, it is considered that the liquid flows to pass through the first gap 61 and the second gap 62 between the joining portions 40 (see FIG. 3). A direction in which the first gap 61 is connected to the second gap 62 is parallel to the direction in which the non-joining portions 50, in which the crush points 53 are formed, are continued. Thus, the liquid may be appropriately diffused by passing through the first gap 61 and the second gap 62.

As illustrated in FIG. 8(a), the number of crush points 53 continuously formed along the one direction may be appropriately adjusted. For example, the number of continuous crush points 53 is preferably three or more, and may be set to a range of 3 to 15 or 5 to 10.

The crush point 53 illustrated in FIGS. 8(a) and 8(b) may be formed in the top sheet according to the first embodiment in addition to the top sheet according to the second embodiment.

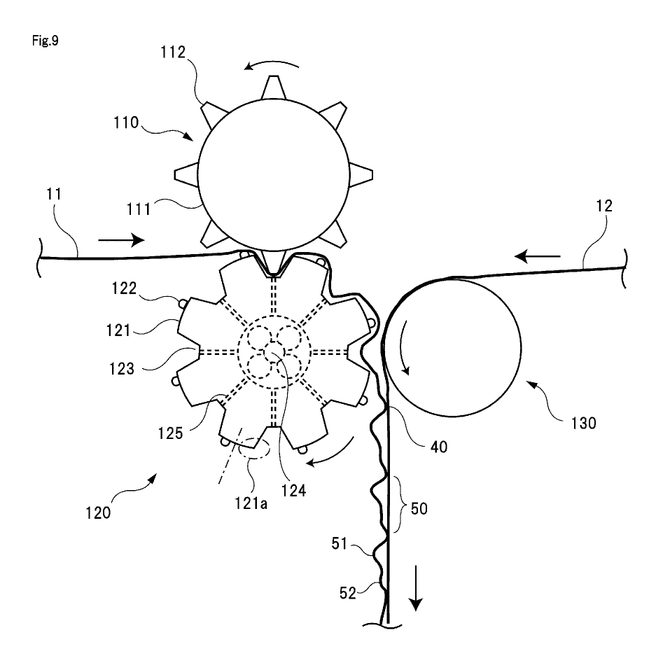

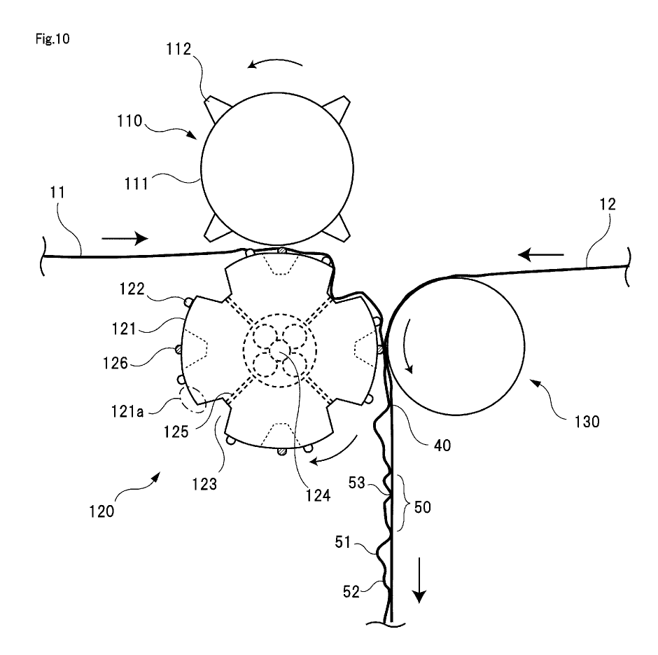

Next, a description will be given of a method of manufacturing the top sheet 10 illustrated in FIGS. 7(a) to 7(c) and FIGS. 8(a) and 8(b) with reference to FIG. 9 and FIG. 10. FIG. 9 and FIG. 10 illustrate a method of forming a convex swelling portion on the upper layer sheet 11 in the upper layer sheet 11 and the lower layer sheet 12 included in the top sheet 10, and then joining the upper layer sheet 11 and the lower layer sheet 12 to each other in a predetermined embossing pattern. As illustrated in FIG. 9 and FIG. 10, an apparatus for manufacturing the top sheet 10 includes a pin roller 110, an embossing roller 120, and a plane roller 130. However, FIG. 9 illustrates a cross-sectional structure of a portion in which the non-joining portion 50 not having the crush point 53 is formed in the pin roller 110, the embossing roller 120, and the plane roller 130. Meanwhile, FIG. 10 illustrates a cross-sectional structure of a portion in which the non-joining portion 50 having the crush point 53 is formed in the pin roller 110, the embossing roller 120, and the plane roller 130.

Firstly, as illustrated in FIG. 9, the pin roller 110 has a convexly projecting projection portion 112 at a plurality of locations on a flat surface 111 included in a peripheral surface thereof. The projection portion 112 of the pin roller 110 is provided mainly to form the convexly swelling large region 51 on the upper layer sheet 11 of the top sheet 10. For this reason, the projection portion 112 is provided at a position that comes into contact with a region scheduled to become the large region 51 of the upper layer sheet 11.

The embossing roller 120 includes a flat hill portion 121 included in a peripheral surface thereof, a plurality of convex embossing protrusions 122 disposed in a predetermined pattern on the hill portion 121, and a plurality of concave hollow portions 123 positioned between hill portions 121. The embossing protrusions 122 are provided to join the upper layer sheet 11 and the lower layer sheet 12 of the top sheet 10 to each other in a predetermined embossing pattern. The embossing protrusions 122 of the embossing roller 120 may be heated by a heating device (not illustrated). In addition, the hollow portions 123 are provided at positions corresponding to the plurality of projection portions 112 provided on the peripheral surface of the pin roller 110, and have shapes capable of receiving the projection portions 112. For this reason, the hollow portions 123 of the embossing roller 120 form the convexly swelling large regions 51 on the upper layer sheet 11 by cooperating with the projection portions 112 of the pin roller 110. The embossing protrusions 122 of the embossing roller 120 are not formed at positions at which the hollow portions 123 are provided.

In addition, as illustrated in FIG. 9, the hill portion 121 is provided between two hollow portions 123 in a rotation direction of the embossing roller 120. In this instance, in the present embodiment, an embossing protrusion 122 is formed at a position close to a hollow portion 123 at a front side of the hill portion 121 in the rotation direction. In other words, the embossing protrusion 122 is formed at a position close to the hollow portion 123 at the front side in the rotation direction in the hollow portion 123 at the front side in the rotation direction and a hollow portion 123 at a rear side in the rotation direction. For this reason, a middle region 121a is present in the hill portion 121 between the embossing protrusion 122 and the hollow portion 123 at the rear side in the rotation direction. The middle region 121a is present at a higher position than the hollow portion 123 and at a lower position than the embossing protrusion 122 when viewed in a radial direction of the embossing roller 120. For this reason, in terms of height, the middle region 121a is provided between the hollow portion 123 and the embossing protrusion 122. The middle region 121 is provided to form the convexly swelling small region 52 on the upper layer sheet 11 of the top sheet 10. Therefore, the middle region 121a is provided at a position adjacent to the hollow portion 123 for forming the large region 51 on the upper layer sheet 11. In an example illustrated in FIG. 9, the embossing protrusion 122 is formed at a position close to the hollow portion 123 at the front side of the hill portion 121 in the rotation direction. However, the embossing protrusion 122 may be formed at a position close to the hollow portion 123 at the rear side of the hill portion 121 in the rotation direction.

In addition, as illustrated in FIG. 9, the embossing roller 120 is preferably connected to a suction device 124. A known device that sucks air using a fan, etc. may be employed as the suction device 124. In addition, as illustrated in FIG. 9, a suction hole 125 of the suction device 124 communicates with a bottom portion of the hollow portion 123 of the embossing roller 120. For this reason, the upper layer sheet 11 pushed into the hollow portion 123 of the embossing roller 120 by the projection portion 112 of the pin roller 110 is partially sucked by the suction device 124 through the suction hole 125. In this way, when a portion of the upper layer sheet 11 pushed into the hollow portion 123 is sucked using the suction device 124, the large region 51 formed on the upper layer sheet 11 may be swelled more.

A peripheral surface of the plane roller 130 corresponds to a smooth surface. The plane roller 130 is provided to interpose the upper layer sheet 11 and the lower layer sheet 12 of the top sheet 10 between the embossing protrusions 122 of the embossing roller 120 and the plane roller 130 to press and heat the sheets, thereby heat-fusing the sheets. The peripheral surface of the plane roller 130 may be made of metal or rubber. In addition, the peripheral surface of the plane roller 130 may be heated by the heating device (not illustrated).

As illustrated in FIG. 9, the pin roller 110 and the embossing roller 120 are disposed to face each other, and the upper layer sheet 11 introduced between both sheets. In addition, the embossing roller 120 and the plane roller 130 are disposed to face each other, and the upper layer sheet 11 and the lower layer sheet 12 are introduced in an overlapping state between both rollers.

Next, an operation of the manufacturing apparatus will be described. As illustrated in FIG. 9, the upper layer sheet 11 drawn from an original fabric roll (not illustrated) is introduced between the pin roller 110 and the embossing roller 120 via one or a plurality of guide rollers (not illustrated). Referring to the upper layer sheet 11, the non-skin facing surface (surface not directly coming into contact with the skin of the wearer) comes into contact with the pin roller 110, and the skin facing surface (surface directly coming into contact with the skin of the wearer) comes into contact with the embossing roller 120. In this instance, the upper layer sheet 11 is fit to the hollow portion 123 of the embossing roller 120 while being pressed against the projection portion 112 of the pin roller 110. At the same time, a portion of the upper layer sheet 11 fit to the hollow portion 123 is sucked by the suction device 124 through the suction hole 125 provided in the bottom portion of the hollow portion 123. In this way, the portion of the upper layer sheet 11 fit to the hollow portion 123 convexly swells. In this way, a portion that swells in a dome shape toward the skin facing surface side, that is, the large region 51 of the non-joining portion 50 is formed on the upper layer sheet 11. Thereafter, the upper layer sheet 11 is introduced between the embossing roller 120 and the plane roller 130 while coming into contact with the peripheral surface of the embossing roller 120.

Meanwhile, the lower layer sheet 12 drawn from another original fabric roll (not illustrated) is introduced between the embossing roller 120 and the plane roller 130 via one or a plurality of guide rollers (not illustrated). The upper layer sheet 11 and the lower layer sheet 12 overlap each other between the embossing roller 120 and the plane roller 130. In this instance, the skin facing surface side of the upper layer sheet 11 comes into contact with the embossing roller 120, and the non-skin facing surface side of the lower layer sheet 12 comes into contact with the plane roller 130. The upper layer sheet 11 and the lower layer sheet 12 are interposed between the embossing roller 120 and the plane roller 130 in a stacked state, and heat-fused by being heated and pressed. In this instance, the upper layer sheet 11 and the lower layer sheet 12 are heat-fused according to an embossing pattern of the plurality of embossing protrusions 122 formed on the peripheral surface of the embossing roller 120. In this way, a plurality of concave joining portions 40 hollowed toward the non-skin facing surface side is formed on the top sheet 10. Meanwhile, the upper layer sheet 11 is not joined to the lower layer sheet 12 in a region corresponding to the middle region 121a positioned between the embossing protrusion 122 and the hollow portion 123 in the embossing roller 120. However, in the region corresponding to the middle region 121a, the upper layer sheet 11 is not fit to the hollow portion 123 of the embossing roller 120. For this reason, in the region corresponding to the middle region 121a, while the upper layer sheet 11 swells to be higher than the joining portion 40, and the upper layer sheet 11 is lower than the large region 51 of the non-joining portion 50. Therefore, a portion slightly swelling in a dome shape toward the skin facing surface side, that is, the small region 52 of the non-joining portion 50 is formed in the region corresponding to the middle region 121a. In this way, in the upper layer sheet 11 and the lower layer sheet 12, a region touching the embossing protrusion 122 of the embossing roller 120 corresponds to the joining portion 40, and a region surrounded by a plurality of joining portions 40 corresponds to the large region 51 or the small region 52 of the non-joining portion 50. In this way, when the upper layer sheet 11 and the lower layer sheet 12 are joined using the embossing roller 120 after the upper layer sheet 11 is pressed by the pin roller 110, the large region 51 and the small region 52 of the non-joining portion 50 may have a dome shape holding a space therein.

Further, a description will be given of a method of forming the non-joining portion 50 having the crush point 53 with reference to FIG. 10. FIG. 10 and FIG. 9 illustrate substantially the same apparatus. However, a cross-section view of FIG. 10 illustrates a cross section of a different portion from that of FIG. 9. As illustrated in FIG. 10, when the crush point 53 is formed in the large region 51 of the non-joining portion 50, a portion of the hollow portion 123 of the embossing roller 120 illustrated in FIG. 9 is eliminated to form the hill portion 121, and a crush point protrusion 126 for forming the crush point 53 is provided in the portion. In other word, in order to form the crush point 53, the hill portion 121 is formed instead of the hollow portion 123 that swells the large region 51, and the crush point protrusion 126 is provided on the hill portion 121 provided instead of the hollow portion 123. In addition, when the hill portion 121 is provided instead of the hollow portion 123, a portion of the projection portion 112 of the pin roller 110 is unnecessary.

The crush point protrusion 126 formed on the peripheral surface of the embossing roller 120 interposes the upper layer sheet 11 and the lower layer sheet 12 between the crush point protrusion 126 and the smooth surface of the plane roller 130, and presses the upper layer sheet 11 toward the lower layer sheet 12 side. In this instance, similarly to the embossing protrusion 122, the crush point protrusion 126 may join (heat-fuse) the upper layer sheet 11 and the lower layer sheet 12 to each other. Alternatively, the crush point protrusion 126 may simply press the upper layer sheet 11 without joining the upper layer sheet 11 and the lower layer sheet 12 to each other. In this way, the crush point 53 at which the upper layer sheet 11 and the lower layer sheet 12 are pressed or joined is formed by the crush point protrusion 126 in the large region 51 of the non-joining portion 50 of the top sheet 10. As described in the foregoing, when the crush point 53 is formed, the upper layer sheet 11 is inhibited from swelling, and thus a space, which is normally formed in the large region 51 of the non-joining portion 50, is narrowed or eliminated. In addition, the shape of the upper layer sheet 11 including nonwoven fabric, etc. is restored to swell around the crush point 53, and thus the small wall portion 54 is formed around the crush point 53 (see FIGS. 8(a) and 8(b)). In this way, according to the apparatus illustrated in FIG. 10, it is possible to form the joining portion 40 for embossing in the upper layer sheet 11 and the lower layer sheet 12, and to form the crush point 53 for controlling the diffusion direction of the liquid at the same time.

### 3. Joining Portion Non-Forming Region

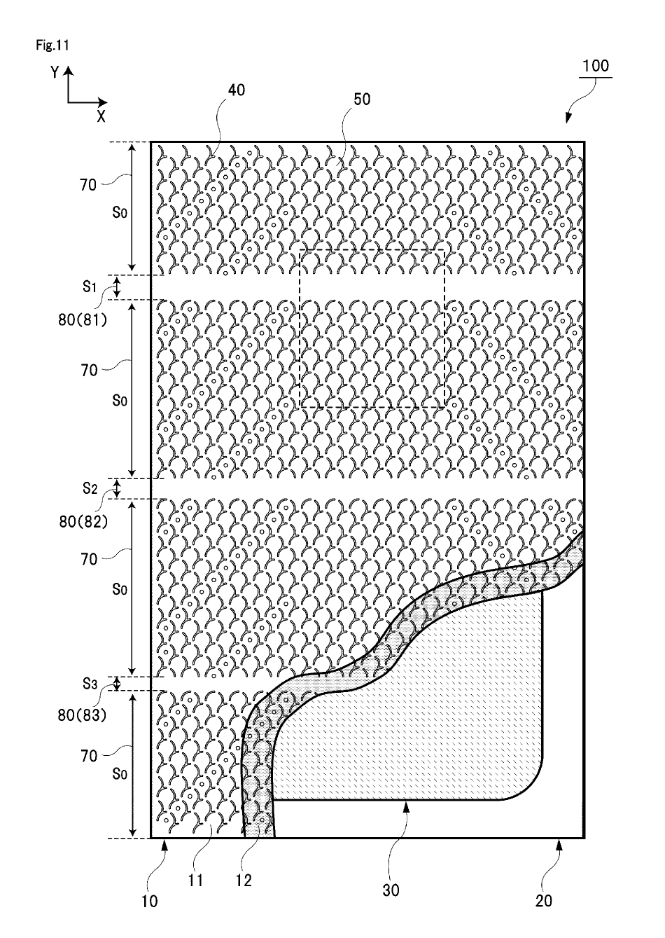

Next, a description will be given of an example of a case in which a joining portion non-forming region 80 is formed in the top sheet 10 of the invention with reference to FIG. 11 to FIG. 13. The joining portion non-forming region 80 described below may be formed in both the first embodiment and the second embodiment described above.

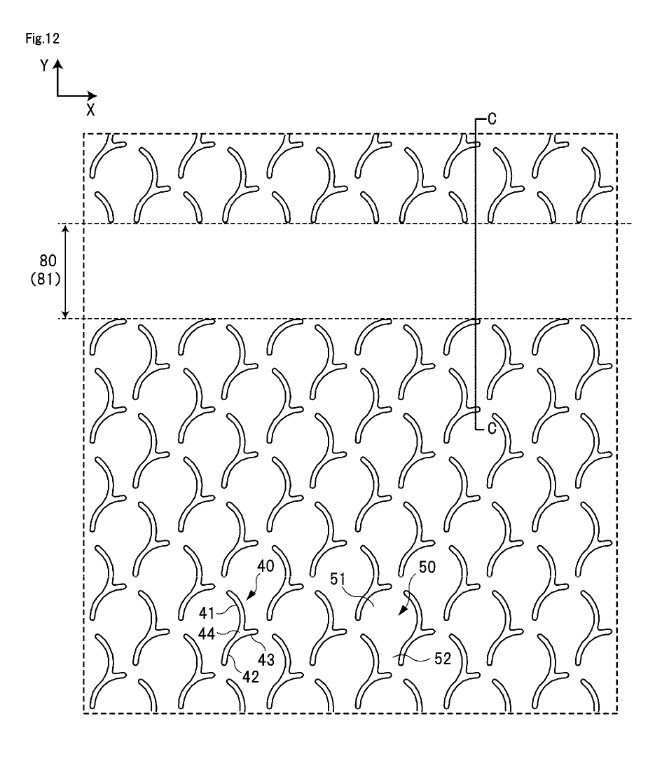

FIG. 11 is an example of a plan view of the top sheet. FIG. 12 is an enlarged view of an inside of a dotted frame illustrated in FIG. 11. As illustrated in FIG. 11, the top sheet 10 is divided into a plurality of joining portion forming regions 70 and a plurality of joining portion non-forming regions 80. The joining portion forming regions 70 are regions in which the above-described joining portions 40 are formed in at least a portion along the width direction (X-axis direction) of the absorbent article. In other words, when a virtual straight line is drawn along the width direction in the joining portion forming regions 70, the joining portions 40 are positioned on the straight line. In the present embodiment, the joining portions 40 are disposed substantially in zigzag, and thus a portion in which the joining portions 40 are regularly formed corresponds to the joining portion forming regions 70. Meanwhile, each of the plurality of joining portion non-forming regions 80 is formed between joining portion forming regions 70. The joining portion non-forming regions 80 are regions in which the above-described joining portions 40 are not formed across the whole in the width direction (X-axis direction) of the absorbent article. In other words, when a virtual straight line is drawn along the width direction in the joining portion non-forming regions 80, no joining portion 40 is positioned on the straight line. In the present embodiment, in order to from the joining portion non-forming regions 80, a portion of the shape of the λ-shaped joining portion 40 is modified such that no joining portion 40 is present across the whole width direction.

In the embodiment illustrated in FIG. 11, the joining portion non-forming regions 80 are provided in three positions. The three respective joining portion non-forming regions 80 are set to a first region 81, a second region 82, and a third region 83. Herein, in FIG. 11, vertical widths (lengths in the longitudinal direction of the absorbent article) of the first region 81, the second region 82, and the third region 83 are indicated by symbols S₁, S₂, and S₃, respectively. Herein, the vertical widths S₁, S₂, and S₃ of the first to third regions 81, 82, and 83 are different from one another. Specifically, the vertical width S₁ of the first region 81 is the widest width, the vertical width S₂ of the second region 82 is the second widest width, and the vertical width S₃ of the third region 83 is the narrowest width (S₁>S₂>S₃). In this way, the plurality of joining portion non-forming regions 80 is preferably formed in at least two or more types of vertical widths, and particularly in three or more types of vertical widths. A joining portion non-forming region 80 having a wide vertical width may smoothly lead urine inside a capillary tube even when the urine is in a state in which salinity concentration and viscosity are high and a movement driving force is small. For this reason, the joining portion non-forming region 80 having the wide vertical width is suitable for diffusion of urine having high salinity concentration. Meanwhile, a joining portion non-forming region 80 having a narrow vertical width may effectively diffuse a liquid in the width direction even when the amount of the liquid is small. Furthermore, the joining portion non-forming region 80 having the narrow vertical width is suitable for diffusion of urine having low salinity concentration. Therefore, it is preferable to provide joining portion non-forming regions 80 having different vertical widths to be able to respond to a change in salinity concentration of urine.

For example, the vertical width S₁ of the first region 81 corresponding to the widest width is preferably set to a range of 6 mm to 10 mm, and particularly preferably set to 8 mm. In addition, the vertical width S₂ of the second region 82 corresponding to the second widest width is preferably set to a range of 4 mm to 8 mm, and particularly preferably set to 6 mm. In addition, the vertical width S₃ of the third region 83 corresponding to the widest width is preferably set to a range of 2 mm to 6 mm, and particularly preferably set to 4 mm. However, a relation of S₁>S₂>S₃ is satisfied.

In addition, in FIG. 11, a vertical width (length in the longitudinal direction of the absorbent article) of the joining portion forming region 70 is indicated by a symbol S₀. The joining portion forming regions 70 are present at a plurality of positions, and vertical widths thereof may be different from each other. However, herein, symbols indicating the vertical widths of the plurality of joining portion forming regions 70 are conveniently uniformly set to S₀. The vertical width S₀ of the joining portion forming region 70 may be twice or more the vertical width S₁ of the first region 81 which is the widest among the plurality of joining portion non-forming regions 80 (81 to 83) (S₀≧2·S₁). In particular, the vertical width S₀ of the joining portion forming region 70 is preferably three times or more the vertical width S₁ of the first region 81, and more preferably five times or seven times the vertical width S₁ of the first region 81. When the vertical width of the joining portion forming region 70 is narrow, it is difficult to obtain softness and air permeability improvement effects resulting from embossing assigned to the joining portion forming region 70. Thus, the vertical width of the joining portion forming region 70, which is wide to some extent, is preferably ensured. Specifically, the vertical width S₀ of the joining portion forming region 70 is preferably 40 mm or more or 60 mm. For example, an upper limit thereof may be set to 100 mm or 150 mm.

In the present embodiment, the joining portion non-forming regions 80 (81 to 83) are arranged such that the vertical widths are in order of S₁-S₂-S₃ (S₁>S₂>S₃) when viewed in the longitudinal direction (X-axis direction). When the plurality of joining portion non-forming regions 80 is arranged such that the vertical widths thereof gradually narrows in the longitudinal direction, it is possible to effectively respond to an individual difference in salinity concentration of urine. However, for example, the joining portion non-forming regions 80 may be arranged in order of S₂-S₁-S₃ or in order of S₁-S₃-S₂ when viewed in the longitudinal direction.

In addition, in the present embodiment, the joining portion non-forming regions 80 different in vertical width are formed only in three positions. However, the number of joining portion non-forming regions 80 may be appropriately adjusted according to a size of the absorbent article, etc. For example, the number of joining portion non-forming regions 80 may be set to five to ten. In this case, all the plurality of joining portion non-forming regions 80 may be different in vertical width, or the joining portion non-forming regions 80 having the same vertical width may be provided in a plurality of positions. For example, when the number of joining portion non-forming regions 80 is six, the joining portion non-forming regions 80 may be arranged by being regularly repeated such that vertical widths correspond to S₁-S₂-S₃-S₁-S₂-S₃ (S₁>S₂>S₃) when viewed in the longitudinal direction (X-axis direction).

In addition, in the present embodiment, the respective joining portion non-forming regions 80 in the three positions are different in vertical width. However, at least one of the vertical widths of the plurality of joining portion non-forming regions 80 may be different. For example, when the joining portion non-forming regions 80 are provided in three positions, vertical widths may be set to S₁-S₂-S₂ (S₁>S₂) when viewed in the longitudinal direction such that a joining portion non-forming region 80 having a wide vertical width (S₁) is provided only in one position, and vertical widths (S₂) in the other two positions may be identical to each other. In addition, for example, when the number of joining portion non-forming regions 80 is six, vertical widths thereof may be set to S₁-S₂-S₂-S₁-S₂-S₂ (S₁>S₂) when viewed in the longitudinal direction.

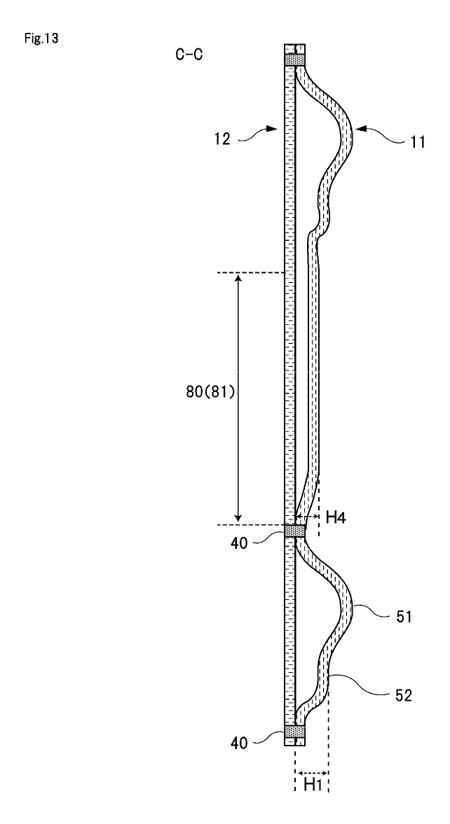

FIG. 13 illustrates a cross-sectional shape along C-C line illustrated in FIG. 12. That is, FIG. 13 illustrates a cross-sectional view of the joining portion non-forming region 80 in the longitudinal direction. As illustrated in FIG. 13, in the joining portion non-forming region 80, the joining portions 40 are not formed, and thus the upper layer sheet 11 and the lower layer sheet 12 are separated from each other. In addition, in FIG. 13, a swelling height of the upper layer sheet 11 in the joining portion non-forming region 80 is indicated by a symbol H₄. In addition, in FIG. 13, a swelling height of the upper layer sheet 11 in the small region 52 described above in the non-joining portion 50 surrounded by the joining portions 40 is indicated by a symbol H₁. In this case, the swelling height H₄ of the upper layer sheet 11 in the joining portion non-forming region 80 is preferably the same as the swelling height H₁ of the upper layer sheet 11 in the small region 52 or lower than the swelling height H₁ (H₄≦H₁). For example, the swelling height H₄ is preferably in a range of 30% to 100%, 40% to 90%, or 50% to 80% with respect to the swelling height H₁. When the swelling height H₄ in the joining portion non-forming region 80 is set to be identical to or preferably set to be lower than the swelling height H₁ in the small region 52 in this way, excreta such as urine, a loose passage, etc. is easily diffused in the width direction through the joining portion non-forming region 80 linearly formed across the whole top sheet 10 in the width direction.

### 4. Characteristic of the Invention from Another Point of View

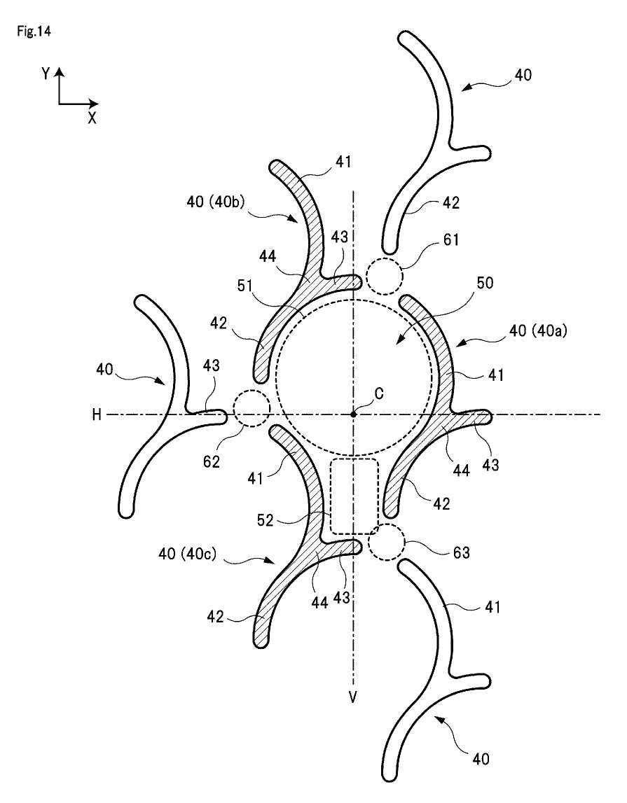

Next, a description will be given of a case in which the top sheet according to the invention is specified from another point of view with reference to FIG. 14 to FIG. 17.

An issue to be solved by the invention described below is to provide a top sheet, absorption performance of which is not impaired in a region in which the top sheet is fully or partially folded, and thus surfaces thereof adhere to each other. In addition, as a result of a keen examination of solution to such an issue, the inventors of the invention have determined to set a shape of a joining portion group positioned around one non-joining region to an asymmetric shape in a regular embossing pattern formed on a surface of the top sheet. When the shape of the joining portion group is set to the asymmetric shape (including front-rear line asymmetry, left-right line asymmetry, or rotational asymmetry) in this way, joining portions may not fully overlap each other even in a state in which the top sheet is fully or partially folded in half, and thus surfaces thereof adhere to each other. In this way, a liquid such as urine may be effectively absorbed even in a region in which the top sheet is folded.

A description will be given of an embossing pattern formed on the top sheet 10. FIG. 14 is an enlarged view illustrating components included in one non-joining portion 50 in the embossing pattern assigned to the top sheet by extracting the components. FIG. 14 is a cross section corresponding to previously described FIG. 3.

As illustrated in FIG. 14, the embossing pattern of the top sheet 10 includes a plurality of joining portions 40 that joins the upper layer sheet 11 and the lower layer sheet 12 to each other, and a plurality of non-joining portions 50 surrounded by the plurality of joining portions 40. The plurality of joining portions 40 is regularly disposed at intervals. Specifically, a plurality of joining portions 40 having the same shape, size, and direction is disposed by being arranged in column shapes at certain intervals in the longitudinal direction, and a plurality of columns of the joining portions 40 is formed in the width direction. In addition, in adjacent columns of joining portions 40, the respective joining portions 40 are differently arranged in the longitudinal direction, and a so-called zigzag arrangement is formed. In addition, the respective joining portions 40 are arranged on straight lines in the longitudinal direction and the width direction.

In the joining portions 40, the upper layer sheet 11 and the lower layer sheet 12 are heat-fused together. For this reason, the upper layer sheet 11 is concavely hollowed in the joining portions 40. Meanwhile, the non-joining portions 50 correspond to regions surrounded by the plurality of joining portions 40, that is, regions in which the upper layer sheet 11 and the lower layer sheet 12 are not joined to each other. For this reason, when compared to the joining portions 40, the non-joining portions 50 convexly swells in a direction in which the upper layer sheet 11 touches the skin of the wearer. As illustrated in FIG. 14, in the top sheet 10 of the invention, the joining portions 40 and the non-joining portions 50 have novel shapes which have not been present in the past.

As illustrated in FIG. 14, in the invention, the plurality of joining portions 40 are disposed around the non-joining portion 50, and the non-joining portion 50 is demarcated in this way. Herein, in this specification, the plurality of joining portions 40 disposed to surround one non-joining portion is collectively referred to as a “joining portion group”. In FIG. 14, a plurality of joining portions 40 included in a certain “joining portion group” is conceptually indicated by a dotted line. In the embodiment illustrated in FIG. 14, the joining portion group includes three joining portions 40.

As illustrated in FIG. 14, in the invention, the joining portion group has an asymmetric shape. Specifically, firstly, a center C of the non-joining portion 50 is determined. The center C of the non-joining portion corresponds to a point at which a straight line which extends along the longitudinal direction and divides a maximum width of the non-joining portion 50 into two equal parts intersects a straight line which extends along the width direction and divides a maximum length of the non-joining portion 50 into two equal parts. Herein, firstly, the joining portion group has a line asymmetric shape, that is, a front-rear line asymmetric shape with respect to a symmetry axis H along the width direction passing through the center C of the non-joining portion 50. That is, even when the joining portion group is folded in half along the symmetry axis H illustrated in FIG. 14, the joining portion group has a shape in which folded parts do not fully overlap in mirror symmetry. Further, the joining portion group has a line asymmetric shape, that is, a left-right line asymmetric shape with respect to a symmetry axis V along the longitudinal direction passing through the center C of the non-joining portion 50. That is, even when the joining portion group is folded in half along the symmetry axis V illustrated in FIG. 14, the joining portion group has a shape in which folded parts do not fully overlap in mirror symmetry.

When the shape of the joining portion group is set to the front-rear line asymmetric shape and the left-right line asymmetric shape in this way, parts of the joining portion 40 may be inhibited from fully overlapping each other even in a state in which the top sheet 10 is fully or partially folded in half in the longitudinal direction or the width direction, and surfaces thereof adhere to each other. In other words, when the top sheet is folded in half in a conventional embossing pattern, recessed joining portions fully overlap each other as in mirror symmetry, and a large gap is generated in the overlapping portion. In addition, referring to the large gap generated on the surface of the top sheet, a liquid such as urine may pass by the gap without being absorbed, or the liquid may accumulate in the gap. Therefore, there has been a problem that there is a concern that absorption performance of the top sheet may be impaired in a region in which the surfaces of the top sheet overlap each other. On the other hand, for example, when the shape of the joining portion group is set to the front-rear line asymmetric shape and the left-right line asymmetric shape as in an example illustrated in FIG. 14, the joining portions 40 may be inhibited from fully overlapping each other even when the top sheet 10 is fully or partially folded in half. Therefore, according to the invention, the liquid such as urine may be effectively absorbed even in a region in which the top sheet 10 is folded.

In addition, as in the example illustrated in FIG. 14, the shape of the joining portion group preferably corresponds to a shape not having rotational symmetry (rotationally asymmetric shape) using the center C of the non-joining portion 50 as a center of symmetry. In this way, not only when the top sheet 10 is folded in half in the longitudinal direction and the width direction, but also when the top sheet 10 is folded in half in an arbitrary direction, the joining portions 40 forming the joining portion group do not fully overlap in mirror symmetry.

Further, all the plurality of joining portions 40 included in the joining portion group preferably has the same shape. Similarly to the previously described shape of the joining portion 40, in the embodiment of FIG. 14, each of the joining portions 40 has a λ-shape branching off in three directions. All the respective joining portions 40 have the same shape, size, and direction, and are regularly arranged in zigzag. In this way, when the shapes of the respective joining portions 40 are identical to each other, the top sheet 10 may be joined without unevenness. In other words, when a plurality of joining portions having different shapes, sizes, and directions is formed, there is a possibility that the upper layer sheet 11 and the lower layer sheet 12 may fail to be fusion-bonded to each other in a joining portion having a complex shape or in a small joining portion. On the other hand, when the shapes of the joining portions 40 are identical to each other, the upper layer sheet 11 and the lower layer sheet 12 may be more reliably fusion-bonded to each other.

In addition, each of the plurality of joining portions 40 included in the joining portion group preferably corresponds to a shape not having rotational symmetry. In a conventional embossing pattern, a joining portion corresponds to a shape having rotational symmetry such as a circular shape, a triangular shape, a rectangular shape, a cross shape, a star shape, etc. On the other hand, as illustrated in FIG. 14, in the invention, the joining portion 40 corresponds to a shape not having rotational symmetry (rotationally asymmetric shape). In this way, even when the top sheet 10 is folded in half along a polygonal line passing through a certain joining portion 40, parts of the joining portion 40 may be inhibited from fully overlapping each other in mirror symmetry. In other words, in the invention, the shape of the joining portion 40 preferably corresponds to a shape in which parts of the joining portion 40 do not overlap each other irrespective of how the joining portion 40 is folded in half.

The top sheet according to the embodiment illustrated in FIG. 14 may basically employ a similar configuration to that of the embodiment described with reference to FIG. 1 to FIG. 13. The description related to FIG. 1 to FIG. 13 may be quoted in a description of a specific structure of the top sheet, and a detailed description will be omitted here.

Hereinafter, a description will be given of a modified example of the top sheet according to the embodiment illustrated in FIG. 14.

### 4-1. First Modified Example

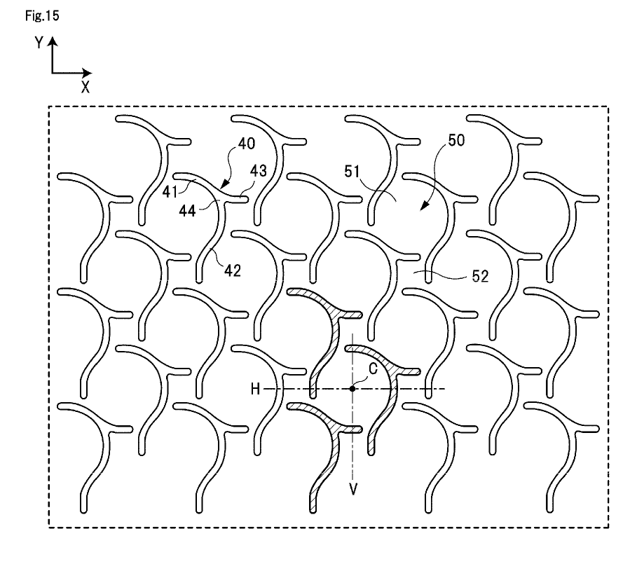

FIG. 15 illustrates a first modified example of the embossing pattern formed on the top sheet. As illustrated in FIG. 15, in the first modified example, a joining portion group including a plurality of joining portions 40 that surrounds one non-joining portion 50 has an asymmetric shape similarly to the embodiment of FIG. 14. In more detail, in Modified Example 1, the joining portion group satisfies requirements below.

(1) The joining portion group is not in line symmetry with respect to the symmetry axis H along the width direction (front-rear line asymmetry).

(2) The joining portion group is not in line symmetry with respect to the symmetry axis V along the longitudinal direction (left-right line asymmetry).

(3) The joining portion group is not in rotational symmetry using the center C of the non-joining portion 50 as a center of symmetry (rotational asymmetry).

(4) All the plurality of joining portions 40 included in the joining portion group is identical to each other.

(5) Each of the plurality of joining portions 40 included in the joining portion group is not in rotational symmetry.

Specifically, as illustrated in FIG. 15, in the embossing pattern according to the first modified example, a joining portion 40 has a shape in which a first line portion 41, a second line portion 42, and a third line portion 43 branch off from a junction 44 thereof in different directions. In other words, proximal ends (one ends) of the first line portion 41, the second line portion 42, and the third line portion 43 are connected at the junction 44, and distal ends (the other ends) of the first line portion 41, the second line portion 42, and the third line portion 43 are directed in different directions. In addition, at least the first line portion 41 and the second line portion 42 have a shape curved in a circular arc shape or an S-shape, or a shape bent at one or more turning points. In the example illustrated in FIG. 15, the first line portion 41 has the circular arc shape, and the second line portion 23 is bent in the S-shape. As illustrated in FIG. 15, referring to the circular arc-shaped first line portion 41 and the S-shaped second line portion 42, directions in which arcs bulge out (directions in which the arcs warp) correspond to the same direction around the junction 44. In other words, when the first line portion 41 and the second line portion 42 are connected to each other, a soft and large S-shape is formed. In addition, the third line portion 43 may have a circular arc shape similarly to the first line portion 41 and the second line portion 42, or have a linear shape. In addition, directions in which the first line portion 41 and the second line portion 42 extend are substantially orthogonal to a direction in which the third line portion 43 extends.

In addition, as illustrated in FIG. 15, the first line portion 41, the second line portion 42, and the third line portion 43 have different lengths. In the example illustrated in FIG. 15, the second line portion 42 is longer than the first line portion 41, and the first line portion 41 is longer than the third line portion 43 (the second line portion 42>the first line portion 41>the third line portion 43). In addition, when all the respective line portions 41 to 43 are formed in circular arc shapes, curvatures (or radii of curvatures) of the respective line portions 41 to 43 may be different from one another or identical to one another.

As illustrated in FIG. 15, the joining portions 40 having the above-described shapes are regularly disposed at intervals. Specifically, a plurality of joining portions 40 having the same shape, size, and direction is disposed by being arranged in column shapes at certain intervals in the longitudinal direction, and a plurality of columns of the joining portions 40 is formed in the width direction. In addition, in adjacent columns of joining portions 40, the respective joining portions 40 are differently arranged in the longitudinal direction, and a so-called zigzag arrangement is formed. In addition, the respective joining portions 40 are arranged on straight lines in the longitudinal direction and the width direction. In this way, the non-joining portion 50 is formed to be surrounded by three joining portions 40. As illustrated in FIG. 15, a substantially circular large region 51 and a substantially rectangular small region 52 are present as the non-joining portion 50. In the first modified example, the joining portion 40 is positioned between the large region 51 and the small region 52. The first modified example is different from the above-described embodiment of FIG. 14 in this regard.

### 4-2. Second Modified Example

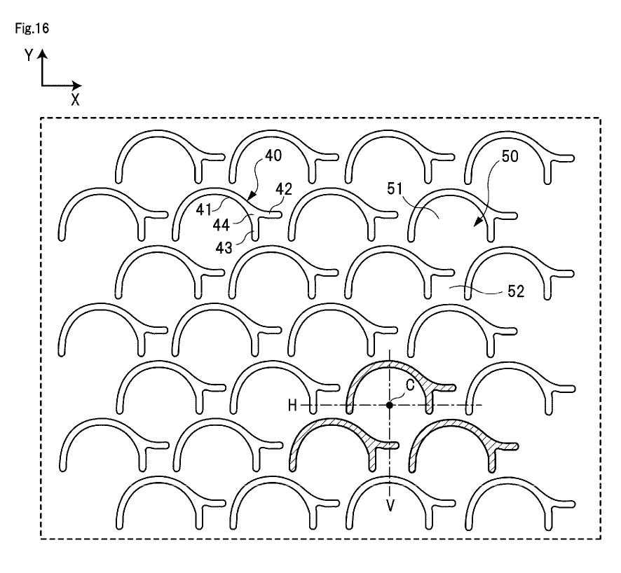

FIG. 16 illustrates an enlarged view of an embossing pattern formed on the top sheet according to a second modified example. As illustrated in FIG. 16, in the second modified example, a joining portion group including a plurality of joining portions 40 that surrounds one non-joining portion 50 has an asymmetric shape similarly to the embodiment of FIG. 14. In more detail, in the second modified example, the joining portion group satisfies requirements below.

(1) The joining portion group is not in line symmetry with respect to the symmetry axis H along the width direction (front-rear line asymmetry).

(2) The joining portion group is not in line symmetry with respect to the symmetry axis V along the longitudinal direction (left-right line asymmetry).

(3) The joining portion group is not in rotational symmetry using the center C of the non-joining portion 50 as a center of symmetry (rotational asymmetry).

(4) All the plurality of joining portions 40 included in the joining portion group is identical to each other.

(5) Each of the plurality of joining portions 40 included in the joining portion group is not in rotational symmetry.

Specifically, as illustrated in FIG. 16, in the embossing pattern according to the second modified example, a joining portion 40 has a shape in which a first line portion 41, a second line portion 42, and a third line portion 43 branch off from a junction 44 thereof in different directions. In other words, proximal ends (one ends) of the first line portion 41, the second line portion 42, and the third line portion 43 are connected at the junction 44, and distal ends (the other ends) of the first line portion 41, the second line portion 42, and the third line portion 43 are directed in different directions. In addition, at least the first line portion 41 has a shape curved in a circular arc shape, or a shape bent at one or more turning points. In the example illustrated in FIG. 16, the first line portion 41 has the circular arc shape. Meanwhile, the second line portion 42 and the third line portion 43 may have a circular arc shape similarly to the first line portion 41, and may have a linear shape. In addition, directions in which the second line portion 42 and the third line portion 43 extend are substantially orthogonal to each other.

In addition, as illustrated in FIG. 16, the first line portion 41, the second line portion 42, and the third line portion 43 have different lengths. Meanwhile, the second line portion 42 and the third line portion 43 have substantially the same length. In the example illustrated in FIG. 16, the first line portion 41 is longer than the second line portion 42 and the third line portion, and the second line portion 42 has substantially the same length as that of the third line portion 43 (the first line portion 41>the second line portion 42=the third line portion 43). In addition, when all the respective line portions 41 to 43 are formed in circular arc shapes, curvatures (or radii of curvatures) of the respective line portions 41 to 43 may be different from one another or identical to one another. In addition, as illustrated in FIG. 16, a substantially semi-circular shape is formed when the first line portion 41 is connected to the third line portion 43.

As illustrated in FIG. 16, the joining portions 40 having the above-described shapes are regularly disposed at intervals. Specifically, a plurality of joining portions 40 having the same shape, size, and direction is disposed by being arranged in column shapes at certain intervals in the longitudinal direction, and a plurality of columns of the joining portions 40 is formed in the width direction. In addition, in adjacent columns of joining portions 40, the respective joining portions 40 are differently arranged in the longitudinal direction, and a so-called zigzag arrangement is formed. In addition, the respective joining portions 40 are arranged on straight lines in the longitudinal direction and the width direction. In this way, the non-joining portion 50 is formed to be surrounded by three joining portions 40. As illustrated in FIG. 16, a substantially circular large region 51 and a substantially rectangular small region 52 are present as the non-joining portion 50. In the second modified example, the joining portion 40 is positioned between the large region 51 and the small region 52. The second modified example is different from the above-described embodiment of FIG. 14 in this regard.

### 4-2. Third Modified Example

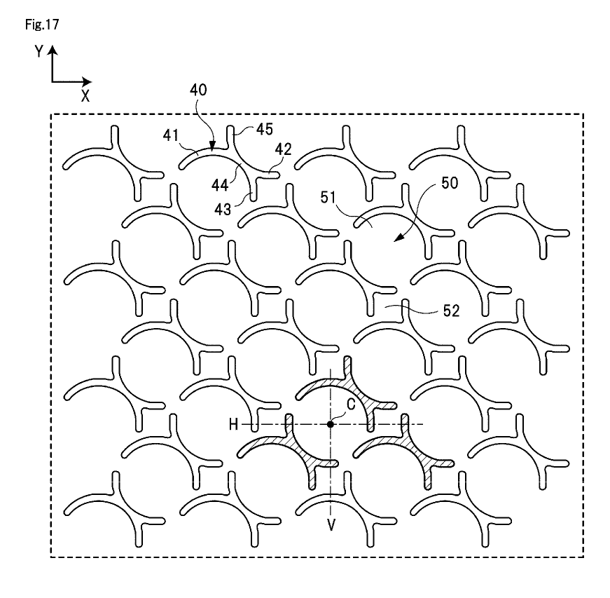

FIG. 17 illustrates an enlarged view of an embossing pattern formed on the top sheet according to a third modified example. As illustrated in FIG. 17, in the third modified example, a joining portion group including a plurality of joining portions 40 that surrounds one non-joining portion 50 has an asymmetric shape similarly to the embodiment of FIG. 14. In more detail, in the third modified example, the joining portion group satisfies requirements below.

(1) The joining portion group is not in line symmetry with respect to the symmetry axis H along the width direction (front-rear line asymmetry).

(2) The joining portion group is not in line symmetry with respect to the symmetry axis V along the longitudinal direction (left-right line asymmetry).

(3) The joining portion group is not in rotational symmetry using the center C of the non-joining portion 50 as a center of symmetry (rotational asymmetry).

(4) All the plurality of joining portions 40 included in the joining portion group is identical to each other.

(5) Each of the plurality of joining portions 40 included in the joining portion group is not in rotational symmetry.

Specifically, as illustrated in FIG. 17, in the embossing pattern according to the third modified example, a joining portion 40 has a shape in which a first line portion 41, a second line portion 42, a third line portion 43, and a fourth line portion 45 branch off from a junction 44 thereof in different directions. In the third modified example, the junction 44 forms a linear shape having a certain length. In addition, the first line portion 41 and the fourth line portion 45 are connected to one end of the linear junction 44, and the second line portion 42 and the third line portion 43 are connected to the other end of the linear junction 44. In addition, at least the first line portion 41 has a shape curved in a circular arc shape, or a shape bent at one or more turning points. In the example illustrated in FIG. 17, the first line portion 41 has the circular arc shape. In addition, the second line portion 42, the third line portion 43, and the fourth line portion 45 may have a circular arc shape or a linear shape. In addition, directions in which the first line portion 41 and the fourth line portion 45 extend are substantially orthogonal to each other, and directions in which the second line portion 42 and the third line portion extend are substantially orthogonal to each other.

In addition, as illustrated in FIG. 17, the first line portion 41, the second line portion 42, the third line portion 43, and the fourth line portion 45 have different lengths. In the example illustrated in FIG. 17, the first line portion 41 is longer than the other line portions 42, 43, and 45, and the other line portions 42, 43, and 45 have substantially the same length (the first line portion 41>the second line portion 42=the third line portion 43=the fourth line portion 45). In addition, when all the respective line portions 41, 42, 43, and 45 are formed in circular arc shapes, curvatures (or radii of curvatures) of the respective line portions may be different from one another or identical to one another.

As illustrated in FIG. 17, the joining portions 40 having the above-described shapes are regularly disposed at intervals. Specifically, a plurality of joining portions 40 having the same shape, size, and direction is disposed by being arranged in column shapes at certain intervals in the longitudinal direction, and a plurality of columns of the joining portions 40 is formed in the width direction. In addition, in adjacent columns of joining portions 40, the respective joining portions 40 are differently arranged in the longitudinal direction, and a so-called zigzag arrangement is formed. In addition, the respective joining portions 40 are arranged on straight lines in the longitudinal direction and the width direction. In this way, the non-joining portion 50 is formed to be surrounded by three joining portions 40. As illustrated in FIG. 17, a substantially circular large region 51 and a substantially rectangular small region 52 are present as the non-joining portion 50. In the third modified example, the joining portion 40 is positioned between the large region 51 and the small region 52. The third modified example is different from the above-described embodiment of FIG. 14 in this regard.

Hereinbefore, in this specification, the embodiments of the invention have been described with reference to the drawings to represent content of the invention. However, the invention is not restricted to the above-described embodiments, and includes a modified mode or an improved mode clear to those skilled in the art based on information described in this specification.

### INDUSTRIAL APPLICABILITY

The invention relates to a top sheet for an absorbent article such as a disposable diaper. For this reason, the invention may be suitably used in a disposable diaper manufacturing industry, etc.

### REFERENCE SIGNS LIST

10 top sheet
11 upper layer sheet
12 lower layer sheet
20 back sheet
30 absorber
40 joining portion
41 first line portion
42 second line portion
43 third line portion
44 junction
50 non-joining portion
51 large region
52 small region
61 first gap
62 second gap
63 third gap
70 joining portion forming region
80 joining portion non-forming region
100 absorbent article
110 pin roller
111 projection portion
120 embossing roller
121 hill portion
121a middle region
122 embossing protrusion
123 hollow portion
124 suction device
125 suction hole
126 crush point protrusion
130 plane roller

1. A top sheet (10) for an absorbent article, comprising:
an upper layer sheet (11);
a lower layer sheet (12);
a plurality of joining portions (40) that joins the upper layer sheet (11) and the lower layer sheet (12) to each other; and
a plurality of non-joining portions (50) surrounded by the plurality of joining portions (40),
wherein each of the non-joining portions (50) includes
a large region (51) having a relatively large area, and
a small region (52) having a relatively small area and extending in one direction from the large region (51), and
the upper layer sheet (11) convexly swells in the large region (51) and the small region (52).

2. The top sheet according to claim 1,
wherein each of the plurality of joining portions (40) has a shape in which a first line portion (41), a second line portion (42), and a third line portion (43) connected to one another branch off from a junction (44) in different directions,
the first line portion (41) is curved or bent to swell in a certain direction, and
the second line portion (42) is curved or bent to swell in an opposite direction to the first line portion (41).

3. The top sheet according to claim 2, wherein three joining portions (40) are positioned around one certain non-joining portion (50), and, when the three joining portions (40) are a first joining portion (40a), a second joining portion (40b), and a third joining portion (40c),
a portion around the one certain non-joining portion (50) is demarcated by
a first line portion (41) and a second line portion (42) of the first joining portion (40a),
a second line portion (42) and a third line portion (43) of the second joining portion (40b),
a third line portion (43) and a first line portion (41) of the third joining portion (40c),
a first gap (61) between the first line portion (41) of the first joining portion (40a) and the third line portion (43) of the second joining portion (40b),
a second gap (62) between the second line portion (42) of the second joining portion (40b) and the first line portion (41) of the third joining portion (40c), and
a third gap (63) between the third line portion (43) of the third joining portion (40c) and the second line portion (42) of the first joining portion (40a).

4. The top sheet according to claim 1, wherein a height at which the large region (51) swells is higher than a height at which the small region (52) swells.

5. The top sheet according to claim 4, wherein the large region (51) and the small region (52) have spaces therein.

6. The top sheet according to claim 4,
wherein the large region (51) has a space therein, and
an inside of the small region (52) is filled with fiber.

7. The top sheet according to claim 1,
wherein crush points (53) that press the upper layer sheet (11) toward a side of the lower layer sheet (12) are formed in some of the plurality of non-joining portions (50), and
the non-joining portions (50) in which the crush points (53) are formed are continuously disposed in a certain direction.

8. The top sheet according to claim 1,
wherein the top sheet (10) is divided into
a plurality of joining portion forming regions (70) in which the joining portions (40) are formed in at least a portion in the width direction, and
a plurality of joining portion non-forming regions (80) in which the joining portions (40) are not formed across the whole width direction between the joining portion forming regions (70),
the plurality of joining portion non-forming regions (80) includes at least
a first region (81) whose length in the longitudinal direction corresponds to a first length (S₁), and
a second region (82) whose length in the longitudinal direction corresponds to a second length (S₂), and
the second length (S₂) is shorter than the first length (S₁).

9. The top sheet according to claim 1, wherein a shape of a joining portion group including a plurality of joining portions (40) that surrounds one non-joining portion (50) is not in line symmetry with respect to a symmetry axis (H) along the width direction passing through a center (C) of the non-joining portion (50).

10. An absorbent article comprising:
a liquid-permeable top sheet (10);
a liquid-impermeable back sheet (20); and
an absorber (30) positioned between the top sheet (10) and the back sheet (20),
wherein the top sheet (10) includes
an upper layer sheet (11),
a lower layer sheet (12),
a plurality of joining portions (40) that joins the upper layer sheet (11) and the lower layer sheet (12) to each other, and
a plurality of non-joining portions (50) surrounded by the plurality of joining portions (40),
each of the non-joining portions (50) includes
a large region (51) having a relatively large area, and
a small region (52) having a relatively small area and extending in one direction from the large region (51), and
the upper layer sheet (11) convexly swells in the large region (51) and the small region (52).

11. The absorbent article according to claim 10,
wherein each of the plurality of joining portions (40) has a shape in which a first line portion (41), a second line portion (42), and a third line portion (43) connected to one another branch off from a junction (44) in different directions,
the first line portion (41) is curved or bent to swell in a certain direction, and
the second line portion (42) is curved or bent to swell in an opposite direction to the first line portion (41).
